(19) United States
(12) Patent Application Publication
(10) Pub. No.: US 2016/0008419 A1
(43) Pub. Date: Jan. 14, 2016

(54) Methods and Compositions for the Prevention and Treatment of Parasitic Disease

(71) Applicant: THE BOARD OF TRUSTEES OF THE LELAND STANFORD JUNIOR UNIVERSITY, Palo Alto, CA (US)

(72) Inventors: Che-Hong Chen, Fremont, CA (US); Kevin Grimes, Stanford, CA (US); Daria Mochly-Rosen, Menlo Park, CA (US)

(21) Appl. No.: 14/773,227

(22) PCT Filed: Mar. 13, 2014
(86) PCT No.: PCT/US14/26444

§ 371 Date: Sep. 4, 2015

Related U.S. Application Data
(60) Provisional application No. 61/783,470, filed on Mar. 14, 2013.

(51) Int. Cl. A61K 38/00; A61K 31/7048; A61K 31/713; A61K 31/16; C12N 15/113; A61K 31/11

(52) U.S. Cl. CPC A61K 38/005; C12N 15/1137; A61K 31/11; A61K 31/713; A61K 31/16; A61K 31/7048

(57) ABSTRACT

Compositions and methods are provided for treating or preventing a parasitic disease in a subject. Aspects of the methods include administering an ALDH antagonist to a subject that is infected with a parasite or that is at risk for being infected with a parasite. Also provided are reagents, devices and kits thereof that find use in practicing the subject methods.

### CROSS REFERENCE TO RELATED APPLICATIONS

Pursuant to 35 U.S.C. §119 (e), this application claims priority to the filing date of the U.S. Provisional Patent Application Ser. No. 61/783,470 filed Mar. 14, 2013; the full disclosure of which is herein incorporated by reference.

### FIELD OF THE INVENTION

This invention pertains to the treatment and prevention of parasitic disease.

### BACKGROUND OF THE INVENTION

Parasitic diseases are caused by pathogens that exploit the host organism—either by leeching off the host for food, or by using the host to replicate—often with harmful consequences. Parasitic worms (helminthes) are the cause of many diseases, including schistosomiasis, lymphaticfilariasis, and onchocerciasis (river blindness). Diseases are also caused by parasitic worms (nematodes) such as the intestinal hookworm, whipworm, and roundworm infections; these three worms together account for over one billion infections. Parasitic protozoa cause a number of other diseases, such as leishmaniasis, African sleeping sickness, Chagas Disease, and malaria. For example, malaria is a life-threatening disease caused by protozoans of the genus Plasmodium that are transmitted to people through the bites of infected Anopheles mosquitoes (FIG. 1). According to WHO, malaria still infects >200 million people and causes >260,000 deaths annually in 2010. Currently, drugs for the prevention and treatment of malaria are still very limited. In addition, parasite resistance to commonly used malaria therapeutics has emerged and presents a constant challenge for the unmet medical needs. The development of new drugs for parasite control and treatment are therefore urgently needed. The present invention addresses these issues.

### SUMMARY OF THE INVENTION

Compositions and methods are provided for treating or preventing a parasitic disease in a subject. Aspects of the methods include administering an ALDH antagonist to a subject that is infected with a parasite or that is at risk for being infected with a parasite. Also provided are reagents, devices and kits thereof that find use in practicing the subject methods.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 depicts the life cycle of malaria parasites. Note that hepatocytes & erythrocytes are the 2 major sites of parasite & host interaction. Most antimalaria drugs targeted blood stage parasites. Taken from the world wide web at the address http:// followed by “microbiology. 4umer.net/t38-the-life-cycle-of-plasmodium-falciparum-malaria-parasite”.

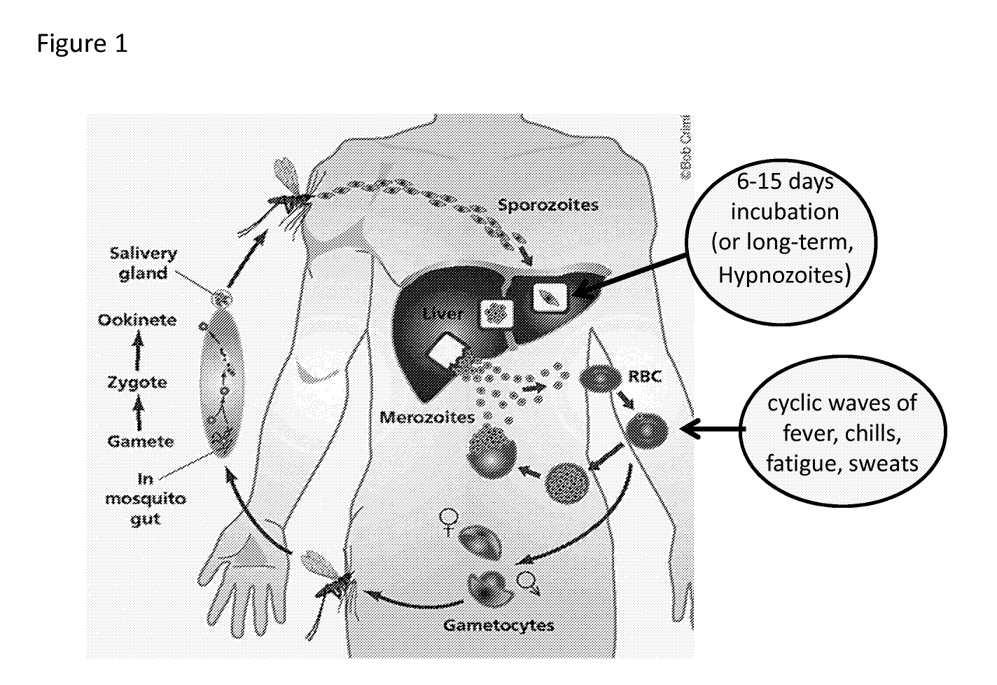

FIG. 2 demonstrates the lack of overlap between global distribution of ALDH2*2 & hemoglobin/G6PD disorders. Shaded grey color indicates areas with high frequencies of sickle cell anemia, thalassemia and G6PD mutations (map combined from (Peters, A. L. et al. Glucose-6-phosphate dehydrogenase deficiency and malaria: cytochemical detection of heterozygous G6PD deficiency in women. J Histochem Cytochem, 2009; 57(11): p. 1003-11; Li H., et al. Refined geographic distribution of the oriental ALDH2*504Lys (nee 487Lys) variant. Ann Hum Genet. 2009; 73 (Pt 3):335-45; WHO, Global distribution of haemoglobin disorders. http://www.who.int/genomics/public/Maphaemoglobin.pdfj). Red outlined area indicates ALDH2*2 prevalence. Each mutation affects a large world population. For example, approximately 350 million people have a deficiency in hemoglobin; approximately 400 million people have a deficiency in G6PD; and approximately 560 million people have a deficiency in ALDH2*2. (Bull. World Health Organ. (1989) 67: 601-611; figures taken from the world wide web at the address http:// followed by “www.who.int/genomics/public/Maphaemoglobin.pdf”, and Li et al. (2009) Annals of Human Genetics 73: 335-345.)

FIG. 3 depicts a phylogenetic tree of the diverse locations of metronidazole-sensitive eukaryotic organisms, constructed by using small ssRNA sequences. An aligned set of ssRNA sequences was downloaded from the web site of the Ribosomal DNA Project, and a parsimony tree was drawn by using the program PAUP (Felsenstein, J. (1989) PHYLIP—phylogeny inference package (version 3.2); Cladistics 5:164-166; Maidak, et al. (1997) The RDP (Ribosomal Database Project). Nucleic Acids Res. 25:109-110; Sohling, B., and G. Gottschalk (1996) Molecular analysis of the anaerobic succinate degradation pathway in Clostridium kluyveri. J. Bacteriol. 178:871-880. Branch lengths have no information in parsimony trees. Organisms containing hydrogenosomes and bacterium-like fermentation enzymes in the cytosol, which are metronidazole sensitive, are indicated to distinguish them from organisms with mitochondria (unmarked) or chloroplasts. The ssRNA tree includes the luminal diplomonad G. lamblia and the free-living diplomonad Hexamita inflata, microsporidia Vairimorpha necatrix and Encephalitozoon hellem, the vaginal trichomonad T. vaginalis and the intestinal trichomonad Dientamoeba fragilis, the microaerophilic ameba E. histolytica and the aerobic amebae Naegleria gruberi and Acanthamoeba castellanii, the slime mold Dictyostelium discoideum, kinetoplastids Leishmania donovani and Euglena gracilis, apicomplexa Plasmodium falciparum and Toxoplasma gondii, anaerobic ciliates Metopus contortus and Dasytricha ruminantium and the aerobic ciliate Tetrahymena pyriformis, plants Glycine max and Arabidopsis thaliana, animals Homo sapiens and Caenorhabditis elegans, and anaerobic fungi Neocallimastix frontalis and Piromonas communis and aerobic fungi Saccharomyces cerevisiae and Candida albicans. A similar tree was obtained by neighbor-joining methods. The closest phylogentic relative of Plasmodium falciparum is Toxoplasma gondii. (Figure taken from: Samuelson (1999) “Why Metronidazole Is Active against both Bacteria and Parasites” Antimicrob & Chemother. 43(7):1533-1541).

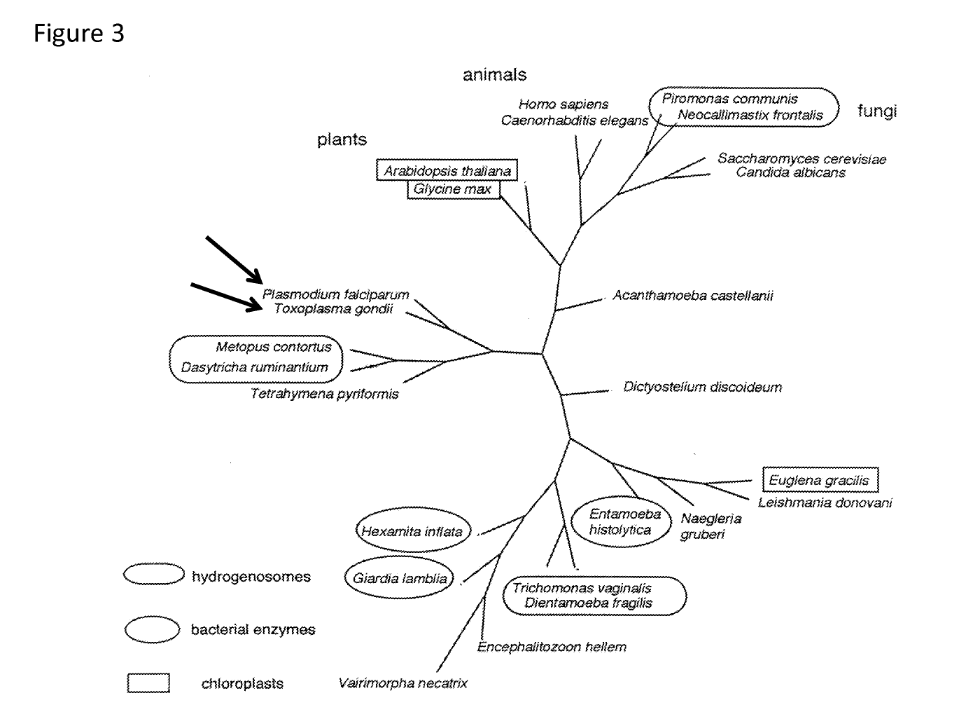

FIG. 4 depicts the family tree of the phylum apicomplexa. Three principal parasitic groups are colored and their life cycle indicated, as well as Cryptosporidium that likely emerged from within gregarines. Numbers on branches and thickness indicates diversity (i.e. named species). © 2011 Jan Slapeta, http://tolweb.org/Apicomplexa.

FIG. 5 demonstrates that no measurable ALDH enzyme activity can be detected in P. falciparum lysates. NADH or NADPH production was monitored in kinetic assays at 340 nm for ALDH and G6PD, respectively. 130 mg of total protein lysate from P. falciparum was used in each assay.

FIG. 6 illustrates how low levels of acetaldehyde reduces parasitemia in P. falciparum infected human red blood cells. Acetaldehyde was diluted in PBS buffer and applied to the parasite culture for 48 hours before the culture medium was replaced with fresh medium. Parasitemia counts were determined by Geimsa stain and FACS.

FIG. 7 depicts two possible scenarios by which accumulation of toxic aldehydes leads to parasite elimination in the liver. One hypothesis relies on the accumulation of endogenous membrane lipid-derived toxic aldehyde during the growth of the sporozoites in hepatocytes. No alcohol consumption is necessary. The other hypothesis relies on the accumulation of alcohol-derived acetaldehyde or other exogenous aldehydes.

FIG. 8 depicts the effect of ALDH inhibitor, disulfiram, on parasitemia in RBC. Parasitemia were determined 24 and 72 hours after the addition of disulfiram at the indicated concentrations.

### DETAILED DESCRIPTION OF THE INVENTION

Compositions and methods are provided for treating or preventing a parasitic disease in a subject. Aspects of the methods include administering an ALDH antagonist to a subject that is infected with a parasite or that is at risk for being infected with a parasite. Also provided are reagents, devices and kits thereof that find use in practicing the subject methods. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods, compositions, and kits are provided for treating or preventing a parasitic disease in an individual. By a “parasitic disease” it is meant a disease caused by or transmitted by a parasite. By a “parasite” it is meant an organism, e.g. a protozoa, a helminthes, an ectoparasite, etc., that lives in or on another organism (its host) and benefits by deriving nutrients at the host's expense. By “treatment”, “treating” and the like it is generally meant obtaining a desired pharmacologic and/or physiologic effect, i.e. treatment or prevention of a parasitic disease. The effect may be prophylactic in terms of completely or partially preventing the parasitic disease or a symptom thereof and/or may be therapeutic in terms of a partial or complete relief from the parasitic disease and/or adverse effects attributable to the parasitic disease. “Treatment” as used herein covers any treatment of a parasitic disease in a mammal, and includes: (a) preventing a parasitic infection in a subject; (b) inhibiting the development of a parasitic disease from an infection, i.e., arresting the development of the parasitic disease in a patient that has been infected with a parasite but has not yet begun to develop symptoms of the parasitic disease; or (c) relieving a parasitic disease, i.e., causing regression of, or relief from, the parasitic disease. The therapeutic agent may be administered before, during or after the onset of the parasitic disease, e.g. before, during or after the infection by the parasite. The treatment of ongoing parasitic disease, where the treatment stabilizes or reduces the parasitic disease of the patient, is of particular interest. The terms “individual,” “subject,” “host,” and “patient,” are used interchangeably herein and refer to any mammalian subject for whom treatment or preventative therapy is desired, e.g. murine, rodent, canine, feline, equine, bovine, ovine, primate, human, etc., particularly human.

In practicing the subject methods, an anti-parasitic composition is administered to a subject, e.g., a subject infected with a parasite associated with the parasitic disease, or a subject at risk of being infected by a parasite associated with the parasitic disease. By an “anti-parasitic composition” or “anti-parasitic agent” it is meant a composition that, when administered in an effective amount, inhibits the effects of a parasite on an individual (i.e. the host), for example, by killing the parasite or the cell infected by the parasite, by preventing the propagation of the parasite, by preventing the production or action of an agent produced by the parasite that is toxic to the individual (i.e. a toxin), etc.

In some embodiments of the subject methods, the anti-parasitic composition comprises or consists essentially of an agent that inhibits, i.e. reduces, or suppresses, aldehyde dehydrogenase activity in the subject. By “aldehyde dehydrogenase activity” it is meant the oxidization (dehydrogenation) of aliphatic and aromatic aldehydes to carboxylic acids in an NAD+- or NADP+-dependent reaction. In some embodiments of the subject methods, aldehyde dehydrogenase activity in a subject or the subject's cells thereof is inhibited by providing an agent that inhibits, i.e. reduces or suppresses, the activity of an aldehyde dehydrogenase (ALDH). In other words, in some embodiments of the subject methods, a subject is administered an effective amount of an ALDH antagonist.

### ALDH Antagonists

Aldehyde dehydrogenases, or “ALDHs”, are a well-known family of aldehyde dehydrogenase enzymes with pyridine-nucleotide-dependent oxidoreductase activity. ALDHs catalyze the oxidation (dehydrogenation) of a wide spectrum of aliphatic and aromatic aldehydic substrates (e.g., a xenogenic aldehyde, a biogenic aldehyde, or an aldehyde produced from a compound that is ingested, inhaled, or absorbed) to carboxylic acids in an NAD⁺- or NADP⁺-dependent reaction. For example, ALDHs oxidizes aldehydes and acetaldehydes derived from the breakdown of compounds, e.g., toxic compounds that are ingested, that are absorbed, that are inhaled, or that are produced as a result of oxidative stress or normal metabolism, e.g., the metabolism of alcohol to acetaldehyde by alcohol dehydrogenase (ADH), the metabolism of retinol to retinal, etc. An aldehyde dehydrogenase can also exhibit esterase activity, i.e. the hydrolysis of esters, and/or reductase activity, e.g. the metabolism of glyceryl trinitrate (GTN) to 1,2-GDN and inorganic nitrite, which results in the formation of NO. An ALDH polypeptide can exhibit one or more of the following enzymatic activities: a) a dehydrogenase activity (e.g., dehydrogenase activity in oxidizing an aldehyde (e.g., a xenogenic aldehyde, a biogenic aldehyde, or an aldehyde produced from a compound that is ingested, inhaled, or absorbed) to the corresponding acid); b) an esterase activity; and c) a reductase activity.

ALDHs may be found in the cytosol, the mitochondria, microsome, and other cellular compartment. Examples of aldehyde dehydrogenases include members of the ALDH1 family, including ALDH1A1 (also known as ALDH1, ALDH-E1, ALDH11, and retinal dehydrogenase 1; see GenBank Accession No. NM_{—}000689); ALDH1A2 (also known as RALDH2 or retinal dehydrogenase 2; see GenBank Accession Nos. NM_{—}003888 (isoform 1), NM_{—}170696.2 (isoform 2), NM_{—}170696.2 (isoform 3), and NM_{—}001206897 (isoform 4)); ALDH1A3 (also known as ALDH6, ALDH1A6, RALDH3, or retinal dehydrogenase 3; see Genbank Accession No. NM_{—}000693); ALDH1B1 (also known as ALDH5 or ALDHX, see GenBank Accession No. NM_{—}000692); ALDH1 L1 (also known as FDH, FTHFD, or cytosolic 10-formyltetrahydrofolate dehydrogenase; see GenBank Accession Nos. NM_{—}001270364 (isoform 1), NM_{—}012190 (isoform 2), and NM_{—}001270365 (isoform 3)); ALDH1L2 (also known as mtFDH or mitochondrial 10-formyltetrahydrofolate dehydrogenase, see GenBank Accession No. NM_{—}001034173); members of the ALDH2 family, in particular ALDH2 (see GenBank Accession Nos. NM_{—}000690 (isoform 1) and NM_{—}001204889 (isoform 2); members of the ALDH3 family, e.g. ALDH3A1 (also known as ALDH3; see GenBank Accession Nos. NM_{—}001135168.1 (variant 1), NM_{—}000691.4 (variant 2), and NM_{—}001135167.1 (variant 3)); ALDH3A2 (also known as ALDH10, FALDH, or fatty aldehyde dehydrogenase; see GenBank Accession Nos. NM_{—}001031806.1 (isoform 1) and NM_{—}000382.2 (isoform 2)); ALDH3B1 (also known as ALDH4; ALDH7; see GenBank Accession Nos. NM_{—}000694.2 (isoform a) and NM_{—}001030010.1 (isoform b)); ALDH3B2 (also known as ALDH8; see GenBank Accession Nos. NM_{—}000695.3 (variant 1) and NM_{—}001031615.1 (variant 2)); members of the ALDH4 family, particularly ALDH4A1 (also known as ALDH4; P5CD; GenBank Accession Nos. NM_{—}003748.3 (isoform a) and NM_{—}001161504.1 (isoform b)); members of the ALDH5 family, particularly ALDH5A1 (also known as SSDH, or succinate-semialdehyde dehydrogenase, mitochondrial; see GenBank Accession Nos. NM_{—}170740.1 (isoform 1) and NM_{—}001080.3 (isoform 2)); members of the ALDH6 family, particularly ALDH6A1 (also known as MMSDH or methylmalonate-semialdehyde dehydrogenase [acylating], mitochondrial; see GenBank Accession No. NM_{—}005589.2); members of the ALDH7 family, particularly ALDH7A1 (see GenBank Accession Nos. NM_{—}001182.4 (isoform 1), NM_{—}001201377 (isoform 2), and NM_{—}001202404 (isoform 3)); members of the ALDH8 family, particularly ALDH8A1 (also known as ALDH12; see GenBank Accession Nos. NM_{—}022568.3 (isoform 1), NM_{—}170771.2 (isoform 2) and NM_{—}001193480.1 (isoform 3); members of the ALDH9 family, particularly ALDH9A1 (also known as E3, ALDH4, ALDH7, ALDH9, TMABADH or 4-trimethylaminobutyraldehyde dehydrogenase; see GenBank Accession No. NM_{—}000696.3); members of the ALDH16 family, particularly ALDH16A1 (see GenBank Accession Nos. NM_{—}153329.3 (isoform 1) and NM_{—}001145396.1 (isoform 2); and members of the ALDH18 family, particularly ALDH18A1 (GSAS, P5CS, PYCS, ARCL3A, or delta-1-pyrroline-5-carboxylate synthase; see GenBank Accession Nos. NM_{—}002860.3 (isoform 1) and NM_{—}001017423.1 (isoform 2)). ALDHs are isozymes, i.e. enzymes that differ in amino acid sequence but catalyze the same chemical reaction. In other words, the enzymes are encoded by different genes, but process or catalyze the same reaction. These enzymes usually display different kinetic parameters (e.g. different KM values), or different regulatory properties. More information regarding the members of the ALDH family of proteins may be found on the world wide web by typing in “www” followed by “aldh.org”.

The term “ALDH” is used herein to encompass any known native ALDH polypeptide or variant/mutant thereof. By “native polypeptide” it is meant a polypeptide found in nature. For example, native ALDH polypeptides include any human ALDH as described herein, the sequences for which may be found at the GenBank Accession Numbers described herein, as well as ALDH homologs that naturally occur in humans and ALDH orthologs that naturally occur in other eukaryotes, e.g. in mice, rodents, canines, cats, equines, bovines, primates. By “variant” or “mutant” it is meant a mutant of the native polypeptide having less than 100% sequence identity with the native sequence. For example, a variant may be a polypeptide having 60% sequence identity or more with a full length native ALDH, e.g. 65%, 70%, 75%, or 80% or more identity, such as 85%, 90%, or 95% or more identity, for example, 98% or 99% identity with the full length native ALDH. Variants also include fragments of a native ALDH polypeptide having aldehyde dehydrogenase activity, e.g. a fragment comprising residues 18-517 of ALDH2 or the comparable sequence in an ALDH homolog or ortholog. Variants also include polypeptides that have aldehyde dehydrogenase activity and 60% sequence identity or more with a fragment of a native ALDH polypeptide, e.g. 65%, 70%, 75%, or 80% or more identity, such as 85%, 90%, or 95% or more sequence identity, for example, 98% or 99% identity with the comparable fragment of the native ALDH polypeptide. Variants may be polypeptides found in nature, or they may be synthetically prepared.

In some embodiments, the agent that inhibits ALDH, i.e. the “ALDH inhibitor”, or “ALDH antagonist”, inhibits a dehydrogenase activity of ALDH. In other words, the agent inhibits the activity of ALDH in oxidizing an aldehyde (e.g., a xenogenic aldehyde, a biogenic aldehyde, or an aldehyde produced from a compound that is ingested, inhaled, or absorbed) to the corresponding acid. In other embodiments, an agent that inhibits ALDH activity inhibits an esterase activity of ALDH. In other embodiments, an agent that inhibits ALDH activity inhibits a reductase activity of ALDH. For example, ALDH can convert nitroglycerin to nitric oxide (NO) via its reductase activity.

In some embodiments, the ALDH antagonist inhibits, i.e. reduces, or suppresses, the enzymatic activity of a particular ALDH. For example, in some embodiments, a subject ALDH antagonist inhibits the enzymatic activity of aldehyde dehydrogenase ALDH2. “ALDH2” or “mitochondrial aldehyde dehydrogenase-2” is a mitochondrial matrix homotetramer aldehyde dehydrogenase with broad specificity and a low Kₘ for acetaldehydes. ALDH2 is a member of the ALDH1B subfamily of ALDHs and is localized to the mitochondrial matrix. Human ALDH2 has a sequence disclosed in GenBank Accession Nos. NM_{—}000690 (isoform 1) and NM_{—}001204889 (isoform 2); a mouse ALDH2 amino acid sequence is found under GenBank Accession No. NP_{—}033786; and a rat ALDH2 amino acid sequence is found under GenBank Accession No. NP_{—}115792. The term “ALDH2” encompasses an aldehyde dehydrogenase that exhibits substrate specificity, e.g., that preferentially oxidizes aliphatic aldehydes. The term “ALDH2” encompasses an enzymatically active polypeptide having at least about 75%, at least about 80%, at least about 85%, at least about 90%, at least about 95%, at least about 98%, at least about 99%, or 100%, amino acid sequence identity to amino acids 18-517 of the amino acid sequence set forth in SEQ ID NO:. The term “ALDH2” as used herein also encompasses fragments, fusion proteins, and variants (e.g., variants having one or more amino acid substitutions, addition, deletions, and/or insertions) that retain ALDH2 enzymatic activity, e.g. 1% or more enzymatic activity, 2% or more enzymatic activity, 5% or more enzymatic activity, 10% or more enzymatic activity, 20% or more enzymatic activity, 30% or more enzymatic activity, 50% or more enzymatic activity, 80% or more enzymatic activity, 90% or more enzymatic activity, or 100% enzymatic activity, i.e. the enzymatic activity of the variant is no different from that of native ALDH2. Enzymatically active ALDH2 variants, fragments, fusion proteins, and the like can be verified by adapting the methods described herein. One example of an ALDH2 variant is ALDH2*2 (SEQ ID NO:2), wherein a lysine residue replaces a glutamate in the active site at position 487 of processed human ALDH2 (residue 504 of unprocessed ALDH2, SEQ ID NO:1), or at a position in a non-human ALDH2 corresponding to amino acid 487 of human ALDH2. This mutation is referred to as the “E487K mutation”; the “E487K variant”; or the “Glu504Lys polymorphism”. See, e.g., Larson et al. (2005) J. Biol. Chem. 280:30550; and Li et al. (2006) J. Clin. Invest. 116:506. Individuals that are homozygous for ALDH2*2 have almost no ALDH2 activity, and those heterozygous for the mutation have reduced activity.

Any agent that inhibits the activity of an ALDH may be employed as an anti-parasitic composition in the subject methods. In other words, any convenient ALDH antagonist, e.g. an ALDH2 antagonist, may be used. Non-limiting examples of ALDH antagonists include agents that reduce the amount of ALDH protein in a cell, e.g. ALDH-specific siRNA, shRNA, antisense RNA, etc.; agents that block the binding of an ALDH to its ligand, e.g. directly, i.e., “competitively”; or indirectly, i.e., “non-competitively”, or “allosterically”, e.g. dominant negative ALDH polypeptides, ALDH-specific antibodies, small molecule inhibitors of ALDH, etc.; and agents that inhibit the activity of proteins upstream of an ALDH, thereby preventing ALDH activation, etc. So, for example, when it is desirable to treat a parasitic disease by administering an agent that inhibits the activity of ALDH2, the subject methods may encompass anti-parasitic agents that reduce the amount of ALDH2 in a cell, e.g. an ALDH2-specific siRNA, shRNA, antisense RNA, etc.; and agents that reduce or suppress the binding of ALDH2 or a variant thereof to its target protein, e.g. by inhibit ALDH2 or variant thereof directly or by inhibiting the activity of proteins upstream of ALDH2, e.g. ALDH2-specific antibodies, dominant negative ALDH2 polypeptides, small molecule inhibitors of ALDH2, etc., as described in greater detail below. Any agent that inhibits the activity of an ALDH may be employed as an anti-parasitic composition in the subject methods. In some instances, two or more ALDH antagonists may be used in the subject methods. In some instances, three or more ALDH antagonists may be used.

As indicated above, one example of a class of ALDH antagonists that may be used as anti-parasitic agents is small molecule inhibitors of ALDH. Naturally occurring or synthetic small molecule compounds of interest include numerous chemical classes, such as organic molecules, e.g., small organic compounds having a molecular weight of more than 50 and less than about 2,500 daltons. The small molecule agent may comprise functional groups for structural interaction with proteins, particularly hydrogen bonding, and typically include at least an amine, carbonyl, hydroxyl or carboxyl group, preferably at least two of the functional chemical groups. The agent may include cyclical carbon or heterocyclic structures and/or aromatic or polyaromatic structures substituted with one or more of the above functional groups. Small molecule agents also include such biomolecules such as peptides, saccharides, fatty acids, steroids, purines, pyrimidines, derivatives, structural analogs or combinations thereof. Non-limiting examples of small molecule ALDH antagonists known in the art include AMPAL (4-amino-4-methyl-2-pentyne-1-al), which inhibits ALDH1 and ALDH3 isozymes; benomyl(methyl-[1-[(butylamino)carbonyl]-1H-benzimidazon-2-yl]carbamate) which inhibits ALDH2 isozymes; calcium carbimide; chloral hydrate (trichloroacetaldehyde monohydrate, which inhibits ALDH2 isozymes; chlorpropamide and analogs thereof (e.g. NPI-1, API-1), which inhibit ALDH2 isozymes; citral (3,7-dimethyl-2,6-octadienal), which inhibits ALDH1, ALDH2, and ALDH3 isozymes; coprine and the coprine metabolite 1-amino cyclopropanol, which inhibit ALDH2 isozymes; cyanamide, which inhibits ADH2 isozymes; antioxidant isoflavones such as daidzin and CVT-10216, which inhibit ALDH2 isozymes; DEAB (4-(diethylamino)benzaldehyde), which inhibits ALDH1 isozymes; daidzin, which inhibits ALDH1 and ALDH2 isozymes; disulfiram (Antabuse; tetraethylthioperoxydicarbonic diamide), which inhibits ALDH1A1 and ALDH2 isozymes; gossypol, which is more specific for ALDH3 isozymes than for ALDH1 and ALDH2 isozymes; kynurenine metabolites, e.g. 3-hydroxykynurenine, 3-hydroxyanthrajnilic acid, kynurenic acid, and indol-3-ylpyruvic acid, which inhibit ALDH2 isozymes; metronidazole; molinate, which inhibits ALDH2; nitrefazole; nitroglycerin (GTN), which inhibits ALDH1, ALDH2, and ALDH3 isozymes; and pargyline (N-benzyl-N-methylprop-2-yn-1-amine), which inhibits ALDH2 isozymes, and the cephalosporin-based antibiotics including, e.g., cefamandole and cefoperazone. See, e.g., Koppaka et al. (2012) Aldehyde Dehydrogenase Inhibitors: a Comprehensive Review of the Pharmacology, Mechanism of Action, Substrate Specificity, and Clinical Application. Pharmacological Reviews 64(3):A-T, the full disclosure of which is incorporated herein by reference. Small molecule compounds can be provided as a solution in DMSO or other solvent.

As indicated above, another example of a class of ALDH antagonists that would be suitable for use as anti-parasitic agents is nucleic acid agents, for example, nucleic acids that encode siRNAs, shRNA, antisense RNA, and the like that target a specific ALDH and inhibit the production of ALDH protein; nucleic acids that encode a dominant negative ALDH polypeptide, e.g. the ALDH2*2 polypeptide, or a dominant negative fragment of an ALDH polypeptide, e.g. a fragment comprising the enzymatic domain of the ALDH2*2 polypeptide; and the like.

Many vectors useful for transferring nucleic acids into target cells are available. The vector may be maintained episomally, e.g. as plasmid, minicircle DNA, virus-derived vector such as cytomegalovirus, adenovirus, etc., or it may be integrated into the target cell genome, through homologous recombination or random integration, e.g. retrovirus derived vectors such as MMLV, HIV-1, ALV, etc. The nucleic acid agent may be provided directly to cells that may be infected by the parasite, or cells at risk for becoming infected by a parasite, e.g. red blood cells or hepatocytes in, e.g., a Plasmodium infection. In other words, the cells are contacted with vectors comprising the nucleic acid of interest such that the vectors are taken up by the cells. Methods for contacting cells with nucleic acid vectors, such as electroporation, calcium chloride transfection, and lipofection, are well known in the art. Alternatively, the nucleic acid agent may be provided to cells via a virus. In other words, the cells are contacted with viral particles comprising the nucleic acid of interest. Retroviruses, for example, lentiviruses, are particularly suitable as vectors for the delivery of an anti-parastic nucleic acid agent. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PAl2 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject cells are targeted by the packaged viral particles. Methods of introducing the retroviral vectors comprising the nucleic acid ALDH antagonist into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription of the ALDH antagonist will be increased above basal levels in the target cell by 5 fold or more, by 10 fold or more, by at least about 100 fold or more, more usually by at least about 1000 fold. In addition, vectors used for providing nucleic acid to the subject cells may include genes that must later be removed, e.g. using a recombinase system such as Cre/Lox, or the cells that express them destroyed, e.g. by including genes that allow selective toxicity such as herpesvirus TK, bcl-xs, etc

As indicated above, a third example of a class of ALDH antagonists that would be suitable for use as anti-parasitic agents is polypeptides, e.g. dominant negative ALDH polypeptides, e.g. the ALDH2*2 polypeptide; dominant negative fragments of ALDH polypeptides, e.g. a fragment comprising the enzymatic domain of the ALDH2*2 polypeptide; proteins that bind specifically to an ALDH and competitively inhibit binding of ligand, e.g. an ALDH-specific antibody; and the like.

Polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Additionally or alternatively, polypeptides may be fused to a polypeptide permeant domain to promote the transport of the polypeptide agent across the cell membrane and into the cell. A number of permeant domains are known in the art and may be used in the polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

A polypeptide agent for use as anti-parasitic agents may be produced by eukaryotic or prokaryotic cells. It may be further processed by unfolding, e.g. heat denaturation, DTT reduction, etc. and may be further refolded, using methods known in the art. The polypeptide may be modified. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. Also included are modifications using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

Whether an agent is an ALDH antagonist can be readily ascertained. Assays for dehydrogenase activity of ALDH are known in the art, and any known assay can be used to detect a suppression of dehydrogenase activity. Examples of dehydrogenase assays are found in various publications, including, e.g., Sheikh et al. ((1997) J. Biol. Chem. 272:18817-18822); Vallari and Pietruszko (1984) J. Biol. Chem. 259:4922; and Farres et al. ((1994) J. Biol. Chem. 269:13854-13860). As an example of an assay for dehydrogenase activity, ALDH aldehyde dehydrogenase activity is assayed at 25° C. in 50 mM sodium pyrophosphate HCl buffer, pH 9.0, 100 mM sodium phosphate buffer, pH 7.4, or 50 mM sodium phosphate buffer, pH 7.4, where the buffer includes NAD+ (e.g., 0.8 mM NAD+, or higher, e.g., 1 mM, 2 mM, or 5 mM NAD+) and an aldehyde substrate such as 14 μM propionaldehyde. Reduction of NAD+ is monitored at 340 nm using a spectrophotometer, or by fluorescence increase using a fluoromicrophotometer. Enzymatic activity can be assayed using a standard spectrophotometric method, e.g., by measuring a reductive reaction of the oxidized form of nicotinamide adenine dinucleotide (NAD+) to its reduced form, NADH, at 340 nm, as described in US 2005/0171043; and WO 2005/057213. In an exemplary assay, the reaction is carried out at 25° C. in 0.1 sodium pyrophosphate (NaPPi) buffer, pH 9.0, 2.4 mM NAD+ and 10 mM acetaldehyde as the substrate. Enzymatic activity is measured by a reductive reaction of NAD+ to NADH at 340 nm, as described in US 2005/0171043; and WO 2005/057213. Alternatively, the production of NADH can be coupled with another enzymatic reaction that consumes NADH and that provides for a detectable signal. An example of such an enzymatic reaction is a diaphorase-based reaction, which reduces resazurin to its oxidized fluorescent compound resorufin, as described in US 2005/0171043; and WO 2005/057213. Detection of fluorescent resorufin at 590 nm provides amplified and more sensitive signals for any change in ALDH aldehyde dehydrogenase enzymatic activity. NADP+ can be used in place of NAD+ in this assay. Suitable substrates include, but are not limited to, octylaldehyde, phenylacetaldehyde, retinaldehyde, and 4-hydroxynonenal. As another example, the effect of a compound on aldehyde dehydrogenase activity of an ALDH polypeptide can be assayed as described in Wierzchowski et al. ((1996) Analytica Chimica Acta 319:209), in which a fluorogenic synthetic substrate, e.g., 7-methoxy-1-naphthaldehyde is used. For example, the reaction could include 7-methoxy-1-naphthaldehyde, NAD+, an ALDH polypeptide, and an ALDH antagonist to be tested; fluorescence (excitation, 330 nm; emission 390 nm) is measured as a readout of enzymatic activity. The dehydrogenase activity of any ALDH polypeptide in the presence of the putative ALDH antagonist can be detected in this manner. The enzyme used in the assay can be purified (e.g., at least about 75% pure, at least about 80% pure, at least about 85% pure, at least about 90% pure, at least about 95% pure, at least about 98% pure, or at least about 99% pure). Recombinant ALDH enzyme can also be used in the assay.

Whether a compound decreases an esterase activity of ALDH can be determined using any known assay for esterase activity. For example, esterase activity of ALDH2 can be determined by monitoring the rate of p-nitrophenol formation at 400 nm in 25 mM N,N-Bis(2-hydroxyethyl)-2-amino ethanesulfonic acid (BES) (pH 7.5) with 800 μM p-nitrophenyl acetate as the substrate at room temperature in the absence or presence of added NAD+. A pH-dependent molar extinction coefficient of 16 mM−1 cm−1 at 400 nm for nitrophenol can be used. See, e.g., Larson et al. (2007) J. Biol. Chem. 282:12940). Esterase activity of ALDH can be determined by measuring the rate of p-nitrophenol formation at 400 nm in 50 mM Pipes (pH 7.4) with 1 mM p-nitrophenylacetate as the substrate. A molar extinction coefficient of 18.3×103 M−1 cm−1 at 400 nm for p-nitrophenolate can be used for calculating its rate of formation. See, e.g., Ho et al. (2005) Biochemistry 44:8022).

Whether a compound decreases a reductase activity of ALDH can be determined using any known assay for reductase activity. A reductase activity of ALDH can be determined by measuring the rate of 1,2-glyceryl dinitrate and 1,3-glyceryl dinitrate formation using a thin layer chromatography (TLC) or liquid scintillation spectrometry method, using a radioactively labeled substrate. For example, 0.1 mM or 1 mM GTN (glyceryl trinitrate) is incubated with the assay mixture (1 ml) containing 100 mM KPi (pH 7.5), 0.5 mM EDTA, 1 mM NADH, 1 mM NADPH in the presence ALDH2. After incubation at 37° C. for about 10 minutes to about 30 minutes, the reaction is stopped and GTN and its metabolites are extracted with 3×4 ml ether and pooled, and the solvent is evaporated by a stream of nitrogen. The final volume is kept to less than 100 ml in ethanol for subsequent TLC separation and scintillation counting. See, e.g., Zhang and Stamler (2002) Proc. Natl. Acad. Sci. USA 99:8306.

In some embodiments, a subject ALDH antagonist is specific for (e.g., selective for) ALDH2, e.g., a subject ALDH2 antagonist decreases an enzymatic activity of an ALDH2 enzyme, but does not substantially decrease the same enzymatic activity of cytosolic aldehyde dehydrogenase-1 (ALDH1), e.g., the subject ALDH2 antagonist decreases an enzymatic activity of an ALDH1 enzyme, if at all, by 15% or less, by 10% or less, by 5% or less, by 2% or less, or by 1% or less, when used at a concentration that decreases the same enzymatic activity of an ALDH2 enzyme by about 5% or more by 10% or more, by 15% or more, by 20% or more, by 25% or more, by 50% or more, by 70% or more, by 80% or more, by 90% or more, or by 100%. In some embodiments, a subject ALDH2 antagonist does not substantially decrease the enzymatic activity of alcohol dehydrogenase (ADH), e.g., a subject ALDH2 antagonist decreases the enzymatic activity of an ADH, if at all, by less than about 5%, less than about 2%, or less than about 1%, when used at a concentration that decreases the enzymatic activity of an ALDH2 enzyme by at least about 5% or more.

For example, in some embodiments, a subject ALDH2 antagonist is specific for (e.g., selective for) ALDH2, e.g., a subject ALDH2 antagonist decreases dehydrogenase activity of an ALDH2 enzyme, but does not substantially decrease the dehydrogenase activity of cytosolic aldehyde dehydrogenase-1 (ALDH1), e.g., a subject ALDH2 antagonist decreases dehydrogenase activity of an ALDH1 enzyme, if at all, by less than about 15%, less than about 10%, less than about 5%, less than about 2%, or less than about 1%, when used at a concentration that decreases dehydrogenase activity of an ALDH2 enzyme by at least about 5% or more. In some embodiments, a subject ALDH2 antagonist does not substantially decrease dehydrogenase activity of alcohol dehydrogenase (ADH), e.g., a subject ALDH2 antagonist decreases the dehydrogenase activity of an ADH, if at all, by less than about 5%, less than about 2%, or less than about 1%, when used at a concentration that decreases the dehydrogenase activity of an ALDH2 enzyme by at least about 5% or more.

In some embodiments, a subject ALDH antagonist decreases an enzymatic activity of certain ALDH enzymes, e.g. the isozymes ALDH1 and ALDH2, but does not substantially decrease the same enzymatic activity of any other ALDH enzyme, e.g., a subject ALDH antagonist decreases an enzymatic activity of an ALDH isozyme other than ALDH1 and ALDH2 by 15% or less, by 10% or less, by 5% or less, by 2% or less, by 1% less, e.g. by a negligible amount, if at all, when used at a concentration that decreases the same enzymatic activity of an ALDH1 and ALDH2 enzyme by at least about 15% or more.

The ALDH antagonist can be incorporated into a variety of formulations. More particularly, the ALDH antagonist may be formulated into pharmaceutical compositions by combination with appropriate pharmaceutically acceptable carriers or diluents.

Pharmaceutical preparations are compositions that include one or more ALDH antagonists present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the ALDH antagonist can be achieved in various ways, including transdermal, intradermal, oral, buccal, rectal, parenteral, intraperitoneal, intradermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For inclusion in a medicament, the ALDH antagonist may be obtained from a suitable commercial source. As a general proposition, the total pharmaceutically effective amount of the ALDH antagonist administered parenterally per dose will be in a range that can be measured by a dose response curve.

ALDH antagonist-based therapies, i.e. preparations of ALDH antagonist to be used for therapeutic administration, may be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The ALDH antagonist-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection. Alternatively, the ALDH antagonist may be formulated into lotions for topical administration.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prevention or treatment of a parasitic disease. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapies that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Methods of Administration

An ALDH antagonist may be administered to an individual by any of a number of well-known methods in the art or described herein for the administration of small molecules, peptides, and nucleic acids to a subject. The ALDH antagonist can be incorporated into a variety of formulations, e.g. as described above or as known in the art. For example, the ALDH antagonist of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the ALDH antagonist can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly parasitic diseases that affect the central nervous system (see, e.g., below), it may be necessary to formulate agents to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversibly affixed (see e.g. US Application Nos. 20080081064 and 20090196903, incorporated herein by reference).

Calculating the effective amount or effective dose of ALDH antagonist to be administered is within the skill of one of ordinary skill in the art, and will be routine to those persons skilled in the art. Needless to say, the final amount to be administered will be dependent upon a variety of factors, include the route of administration, the nature of the parasitic disease that is to be treated, the health and physical condition of the individual to be treated, age, the taxonomic group of individual to be treated (e.g., human, non-human primate, primate, etc.), and factors that will differ from patient to patient. A competent clinician will be able to determine an effective amount of a therapeutic agent to administer to a patient to prevent the development or, or halt or reverse the progression of, the parasitic disease condition as required. Utilizing LD₅₀ animal data, and other information available for the agent, a clinician can determine the maximum safe dose for an individual, depending on the route of administration. For instance, an intravenously administered dose may be more than an intrathecally or topically administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Individual doses are typically not less than an amount required to produce a measurable effect on the individual, and may be determined based on the pharmacokinetics and pharmacology for absorption, distribution, metabolism, and excretion (“ADME”) of the ALDH antagonist or of its by-products, and thus based on the disposition of the composition within the subject. This includes consideration of the route of administration as well as dosage amount, which can be adjusted for topical (applied directly where action is desired for mainly a local effect), enteral (applied via digestive tract for systemic effects, or local effects when retained in part of the digestive tract), or parenteral (applied by routes other than the digestive tract for systemic or local effects) applications. For instance, administration of the ALDH antagonist may be via injection, e.g. intravenous, intramuscular, intracranial, or intraventricular injection, or a combination thereof.

The ALDH antagonist may be administered by infusion or by local injection, e.g. by infusion at a rate of about 50 mg/h to about 400 mg/h, including about 75 mg/h to about 375 mg/h, about 100 mg/h to about 350 mg/h, about 150 mg/h to about 350 mg/h, about 200 mg/h to about 300 mg/h, about 225 mg/h to about 275 mg/h. Exemplary rates of infusion can achieve a desired therapeutic dose of, for example, about 0.5 mg/m²/day to about 10 mg/m²/day, including about 1 mg/m²/day to about 9 mg/m²/day, about 2 mg/m²/day to about 8 mg/m²/day, about 3 mg/m²/day to about 7 mg/m²/day, about 4 mg/m²/day to about 6 mg/m²/day, about 4.5 mg/m²/day to about 5.5 mg/m²/day. Administration (e.g., by infusion) can be repeated over a desired period, e.g., repeated over a period of about 6 hours, about 12 hours, about 24 hours, or about 48 hours to about once every several days, for example, about every five days, etc. It also can be administered prior, at the time of, or after other therapeutic interventions, e.g. the administration of other anti-parasitic drugs, the administration of therapeutics directed at treating symptoms of the parasitic disease, etc. The ALDH antagonist can also be administered as part of a combination therapy, in which one or more other anti-parasitic agents or agents to treat the symptoms of the parasitic disease is also administered to the subject.

Disposition of the ALDH antagonist and its corresponding biological activity within a subject is typically gauged against the fraction of ALDH antagonist present at a target of interest. Thus dosing regimens in which the ALDH antagonist is administered so as to accumulate in a target of interest over time can be part of a strategy to allow for lower individual doses. This can also mean that, for example, the doses of ALDH antagonist that are cleared more slowly in vivo can be lowered relative to the effective concentration calculated from in vitro assays (e.g., effective amount in vitro approximates mM concentration, versus less than mM concentrations in vivo).

As an example, the effective amount of an ALDH antagonist can be gauged from the EC₅₀ of a given ALDH antagonist concentration. By “EC₅₀” is intended the plasma concentration required for obtaining 50% of a maximum effect in vivo. In related embodiments, dosage may also be determined based on ED₅₀ (effective dosage).

In general, with respect to the subject methods, an effective amount of ALDH antagonist is usually not more than 100× the calculated EC₅₀. For instance, the amount of a ALDH antagonist that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC₅₀. The effective amount may be about 1× to 30× of the calculated EC₅₀, and sometimes about 1× to 20×, or about 1× to 10× of the calculated EC₅₀. The effective amount may also be the same as the calculated EC₅₀ or more than the calculated EC₅₀. The EC₅₀ can be calculated by modulating the enzymatic activity of the ALDH polypeptide, e.g. the aldehyde dehydrogenase activity, in vitro. The procedure can be carry out by methods known in the art.

Effective dose regimens can readily be determined empirically from assays, from safety and escalation and dose range trials, individual clinician-patient relationships, as well as in vitro and in vivo assays such as those described herein and illustrated in the Experimental section, below. For example, if a concentration used for carrying out the subject method in mice ranges from about 1 mg/kg to about 25 mg/kg based on the body weight of the mice, an example of a concentration of the ALDH antagonist that can be employed in human may range about 0.083 mg/kg to about 2.08 mg/kg. Other dosage may be determined from experiments with animal models using methods known in the art (Reagan-Shaw et al. (2007) The FASEB Journal 22:659-661).

Typically, the ALDH antagonist is provided to cells in a therapeutically or prophylactically effective amount. By “a therapeutically effective amount”, “a prophylactically effective amount” or “an effective amount” it is meant an amount of an agent that, when administered to a mammal or other subject for treating the parasitic disease, is sufficient, either alone in one or more doses, or in combination in one or more doses with another agent, to halt development of symptoms of the parasitic disease, and in some instances to relieve the symptoms of the parasitic disease. The “therapeutically effective amount” will vary depending on the compound, the parasite, and its severity and the age, weight, etc., of the individual to be treated.

For example, an effective amount of an ALDH antagonist is the dose that, when administered for a suitable period of time, usually 1-10 days, e.g. 1 day or more, 2 days or more, 3 days or more, 4 days or more, in some cases 5 days or more, 6 days or more, 7 days or more, occasionally for 9 or 10 days, will evidence an alteration in the symptoms of the parasitic disease. For example, a therapeutically effective amount or effective dose of an ALDH antagonist (e.g. ALDH2 antagonist) is the dose that, when administered for a suitable period of time, usually at least about 1 day or more, e.g. usually 1-10 days, e.g. 1 day or more, 2 days or more, 3 days or more, 4 days or more, in some cases 5 days or more, 6 days or more, 7 days or more, occasionally for 9 or 10 days, will decrease symptoms associated with the parasitic disease by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100% (or two-fold). For example, if the parasitic disease is malaria, an effective amount of ALDH antagonist may be expected to decrease one or more of the symptoms associated with malaria, e.g. fever, chills, headache, sweats, fatigue, nausea, dry (nonproductive cough), muscle and/or back pain, an enlarged spleen, etc. by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100% (or two-fold). In some instances, the ALDH antagonist may be prophylactically therapeutic, e.g. the therapeutically effective amount will be the amount sufficient to prevent a parasitic disease, for example when delivered before infection by the parasite, or after infection by the parasite but prior to the development of symptoms of the parasitic disease. It will be understood by those of skill in the art that this effect may be achieved by a single dose or by multiple doses.

The therapeutically effective dose may be readily determined using any convenient preclinical or clinical assay e.g. as known in the art or described herein. For example. symptoms of the parasitic disease such as those described herein may be assessed in an animal administered an ALDH antagonist. For example, if the parasitic disease is malaria, and the individual has symptoms such as fever, chills, headache, sweats, fatigue, anemia, nausea, dry (nonproductive) cough, muscle and/or back pain, an enlarged spleen, hemolytic anemia, kidney failure, liver failure, meningitis, pulmonary edema, or hemorrhaging from the spleen, the effect of the ALDH antagonist on one or more of these symptoms may be assessed, e.g. by assessing body temperature (to detect an effect on fever), assessing the red blood cell count (to detect an effect on the anemia), palpating the abdomen (to detect an an effect on the enlarged spleen), measuring protein in a urine sample (to detect an effect on the progression of kidney dysfunction), performing liver function tests (LFTs) on a blood sample to measure indicators of liver failure, e.g., albumin, alanine transaminase, Alkaline phosphatase, bilirubin, gamma glutamyl transpeptidase (to detect an effect on the progression of liver dysfunction), and performing a chest radiography (to detect an effect on the pulmonary edema). Such results are typically compared to the results from a control, or reference, sample, e.g. an animal not administered the ALDH antagonist. In some instances, the method further comprises the step of measuring one or more of these symptoms before and after administration of the ALDH antagonist.

Biochemically speaking, an therapeutically effective amount or effective dose of an ALDH antagonist will be the amount required to decrease the enzymatic activity of an ALDH polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or in some instances by about 100%, i.e. to negligible levels, when compared to the enzymatic activity of the ALDH polypeptide in the absence of the antagonist. In certain embodiments, the ALDH polypeptide is an ALDH2 polypeptide or variant thereof.

In some embodiments, an effective amount of a subject ALDH antagonist is the amount effective to decrease a dehydrogenase activity (e.g., dehydrogenase activity in oxidizing an aldehyde (e.g., a xenogenic aldehyde, a biogenic aldehyde, or an aldehyde produced from a compound that is ingested, inhaled, or absorbed) to the corresponding acid) of an ALDH polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, in some instances by about 100%, i.e. to negligible levels when compared to the dehydrogenase activity of the ALDH polypeptide in the absence of the antagonist. In certain embodiments, the ALDH polypeptide is an ALDH2 polypeptide or variant thereof.

In some embodiments, an effective amount of a subject ALDH antagonist is the amount effective to decrease the esterase activity of an ALDH polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, in some instances by about 100% when compared to the esterase activity of the ALDH polypeptide in the absence of the antagonist. In certain embodiments, the ALDH polypeptide is an ALDH2 polypeptide or variant thereof.

In some embodiments, an effective amount of a subject ALDH antagonist is the amount effective to decrease the reductase activity of an ALDH polypeptide by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, in some instances by about 100%, i.e. to negligible amounts, when compared to the reductase activity of the ALDH polypeptide in the absence of the antagonist. In certain embodiments, the ALDH polypeptide is an ALDH2 polypeptide or variant thereof.

The extent to which ALDH enzymatic activity is modulated by an ALDH antagonist can be readily determined by any convenient method, e.g. as known in the art or as described herein. For example, ALDH enzymatic activity may be determined spectrophotometrically by monitoring the reductive reaction of NAD+ to NADH at A340 nm in the presence of acetaldehyde. As another example, the presence and concentration of aldehyde adducts, e.g. 4-Hydroxinonenal (4-HNE) protein adducts, in tissue may be assessed by Western blotting using an antibody specific for HNE amino acid adducts (Calbiochem, NJ) In this way, the antagonistic effect of the agent may be confirmed.

In some embodiments, the agent that inhibits ALDH activity may be administered alone, e.g. in the absence of other therapeutic agents. In other embodiments, the ALDH antagonist may be administered in combination with other agents, e.g. other anti-parasitic agents, e.g. toxic aldehydes (e.g. 4-Hydroxy-2-nonenal (4-HNE), acetaldehyde), chloroquine, quinidine, doxycycline, tetracycline, clindamycin, atovaquone plus proguanil (Malarone), Mefloquine, artesunate, and pyrimethamine plus sulfadoxine (Fansidar), etc., or may be administered in conjunction with other therapies, e.g. surgical interventions. For example, the subject methods may comprise the step of administering an effective amount of one or more additional anti-parasitic agents. In some instances, the ALDH antagonist may be administered before the one or more additional parasitic agents. In some instances, the ALDH antagonist may be administered after the one or more additional parasitic agents. In some instances, the ALDH antagonist may be administered concurrently with the one or more additional parasitic agents.

In some embodiments, the method further comprises the step of determining if the subject is infected with a parasite. In some such embodiments, this determination step occurs prior to administering the ALDH antagonists, and the administering of the ALDH antagonist is based upon the result of the determining step. Methods for determining if an individual has been infected with a parasite are well known in the art. For example, an individual may be diagnosed as being infected with a Plasmodium, Babesia, or Theileria by microscopic examination of blood using blood films, with antigen-based Rapid Diagnostic Tests (RDT), e.g., immunochromatography-based RDTs, by detection of parasite DNA by polymerase chain reaction (PCR), etc. Any convenient method may be used to determine if the individual has been infected with a parasite.

As an alternative to, or in addition to, administering an ALDH antagonist to a subject that is infected with a parasite or that is at risk of being infected with a parasite (i.e., the host, e.g., human, livestock, etc.), an ALDH antagonist can be administered to the vector that transmits the parasite to the subject. By a “vector” it is meant an organism that carries a disease-causing agent and transmits the agent to a host. Many parasitic diseases are transmitted in this way. Nonlimiting examples of vectors include mosquitoes, flies, sand flies, lice, fleas, ticks and mites. For example, the disease-causing parasite (Plasmodium) that causes malaria is transmitted to humans (the host) by mosquitoes (the vector). As another non-limiting example, disease-causing parasites (Borrelia), which cause Lyme disease, are transmitted to humans (the host), by ticks (the vector). Because an ALDH antagonist will function regardless of the biological context (e.g., whether the target disease-causing agent resides in a host or in a vector), it is possible to prevent or reduce infection of the host by administering an ALDH antagonist directly to a vector.

Methods of administering an ALDH antagonist to a vector will be well within the skill set and knowledge of one of ordinary skill in the art. Representative methods of administration include spraying a vector (e.g., a mosquito, a tick, a population of mosquitoes, a population of ticks, etc.) with an aerosol formulation comprising an ALDH antagonist, adding an ALDH antagonist to the water supply consumed by a vector, and providing to a vector (e.g., a mosquito, a tick, a population of mosquitoes, a population of ticks, etc.) an attractant or food source comprising an ALDH antagonist.

In some cases, a vector (e.g., a mosquito, a tick, etc.) may be genetically modified to express an ALDH antagonist or genetically modified such that it ceases to express ALDH (e.g., an ALDH gene knockout). Vectors that are genetically modified can then be introduced into the vector population (e.g., mosquito population, tick population, etc.) using any convenient population replacement strategy, which will be readily understood by one of ordinary skill in the art. For examples, see Lambrechts et al., J Virol. 2012 February; 86(3):1853-61. doi: 10.1128/JVI. 06458-11; Marshall et al., J Hered. 2011 May-June; 102(3):336-41; Marshall et al., Genetics. 2011 February; 187(2):535-51. doi: 10.1534/genetics. 110.124479; Magori et al., PLoS Negl Trop Dis. 2009 September 1; 3(9):e508; and Labbé et al., Genetics. 2009 May; 182(1):303-12, the disclosures of which are herein incorporated by reference in their entirety.

### Utility

The subject methods and compositions find use in treating or preventing parasitic disease in any mammal for whom treatment or preventative therapy is desired, e.g. murine, rodent, canine, feline, equine, bovine, ovine, primate, human, etc., particularly human.

In some instances, the subject is infected with the parasite. In such instances, the subject methods may find use in preventing or treating a parasitic disease. Any convenient method, e.g. as described herein or known in the art, may be used to determine if the individual has been infected with a parasite. In some embodiments, the method further comprises the step of determining if the individual has a parasite infection, e.g. by detecting a parasite in the individual.

In other instances, the subject is an individual that is at risk for being infected with a parasite. In such instances, the subject methods may find use in preventing a parasitic disease. Mammals can become infected with parasites from contaminated food or water, bug bites, or sexual contact. Parasites may enter the body through the skin or mouth. As such, risk factors for being infected with a parasite includes, for example, whether the subject is likely to be exposed to the parasite, e.g., where the subject is planning on traveling, whether the subject is likely to come into contact with carriers, e.g. insects, animals, people, plants, etc., that may transmit the pathogen, etc. Other non-limiting risk factors include the age of the subject; and whether the subject is immunocompromised, e.g. due to therapy or disease. These risk factors are well known in the art and can be readily assessed by the ordinarily skilled artisan. In some embodiments, the method further comprises the step of determining if the individual is at risk for being infected by a parasite infection.

In some instances, the parasitic disease is caused by a protozoan. By a protozoan, it is meant a unicellular organism of about 1 μm-1 mm in size, in some instances having flagella, cilia or pseudopodia. Non-limiting examples of parasitic protozoans include Entamoeba histolytica, Babesia, Theileria, Balantidium coli, Trypanosoma cruzi, Apicomplexa, Giardia lamblia, Leishmania, Plasmodium, Trypanosoma brucei, and Toxoplasma gondii. In certain instances, the protozoan is a hematozoan, e.g. Plasmodium, Babesia, Theileria. In other instances, the parasitic disease is caused by a helminth. By a helminth, it is meant a parasitic worm, i.e. a worm-like organism that lives in and feeds on a living hosts, receiving nourishment and protection while disrupting their hosts' nutrient absorption. Examples of helminthes include nematodes (roundworms, including hookworms, pinworms, and whipworms), trematodes (flukes) and cestodes (tapeworms). Non-limiting examples of parasitic helminths include the nematodes Ascaris lumbricoide, Baylisascaris procyonis, Dracunculus, Enterobius vermicularis, Gnathostoma spinigerum, Gnathostoma hispidum, Onchocerca volvulus, Strongyloides stercoralis, and Trichuris trichiura; the trematodes Fasciola hepatica, Fasciola gigantica, Fasciolopsis buski, Metagonimus yokagawai, Metagonimus takashii, Metagonimus miyatai, Schistosoma; and the cestodes Diphyllobothrium, Echinococcus, Hymenolepis nana, Hymenolepis diminuta, and Taenia.

Nonlimiting examples of parasitic diseases in humans that may be treated by the methods and compositions of the present invention include:
Acanthamoeba keratitis, an infection of the cornea of the eye by the protozoa Acanthamoeba, characterized by corneal ulcers and even blindness;
Amoebiasis, a protozoan disease of the gastrointestinal tract caused by the amoeba Entamoeba histolytica, characterized by diarrhea or dysentery with blood and mucus in the stool;
Ascariasis, caused by the nematode Ascaris lumbricoides, presenting with symptoms including visceral damage, peritonitis and inflammation, enlargement of the liver or spleen, and a verminous pneumonitis;
Babesiosis, a malaria-like parasitic disease in mammals caused by infection with the protozoa Babesia (e.g., B. microti in humans, B. canis rossi and B. canis canis in dogs, B. bovis in cows, and B. bigemina in cattle); characterized by symptoms ranging from mild fever and diarrhea to high fever, shaking chills, and severe anemia;
Balantidiasis, caused by infection with the protozoa Balantidium coli, characterized by symptoms that may include diarrhea or constipation;
Baylisascariasis, a parasitic disease of the central nervous system caused by infection with the nematode Baylisascaris procyonis, characterized by severe neurological defects;
Chagas Disease, a tropical parasitic disease caused by the flagellate protozoan Trypanosoma cruzi, which may be asymptomatic or characterized by fever, fatigue, body aches, headache, rash, loss of appetite, diarrhea, or vomiting in the acute phase, and is associated with cardiac damage including dilated cardiomyopathy, or digestive system damage resulting in dilation of the digestive tract (megacolon and megaesophagus) and swallowing difficulties and/or severe weight loss in the chronic phase;
Clonorchiasis, caused by the Chinese liver fluke Clonorchis sinens, which presents with symptoms of jaundice in the eyes and skin, enlargement and tenderness of the liver, nausea, diarrhea, abdominal pain, and loss of appetite;
Cochliomyia, caused by the larvae of the blowfly Cochliomyia, which produces deep, pocket-like lesions in the skin;
Cryptosporidiosis, a gastrointestinal disease caused by Cryptosporidium, a protozoan parasite in the phylum Apicomplexa; it is typically an acute short-term infection;
Diphyllobothriasis, caused by the cestode Diphyllobothrium, characterized by symptoms such as irritability, tingling and numbness of the skin, increased heart rate, muscular weakness, and/or abdominal discomfort;
Dracunculiasis, a nodular dermatosis produced by the development of the nematode Dracunculus in the subcutaneous tissue of mammals, characterize by a painful, burning sensation beneath the skin;
East Coast fever, a disease of cattle, sheep and goats caused by the protozoan parasite T. parva, characterized by fever and enlarged lymph nodes near the tick bite(s), anorexia, dyspnea, corneal opacity, nasal discharge, frothy nasal discharge, diarrhea, pulmonary edema, leukopenia, and anemia;
Echinococcosis, caused by the larval stages of different species of cestodes of the genus Echinococcus, which develop cysts in the liver, lung, spleen, brain, heart and kidneys;
Elephantiasis, a parasitic disease of the lymphatic system caused by thread-like parasitic worms such as Wuchereria bancrofti, Brugia malayi, and B. timori, all of which are transmitted by mosquitoes; the disease itself is a result of the interplay between the worm, the symbiotic Wolbachia bacteria within the worm, and the host's immune response, and is characterized by the thickening of the skin and underlying tissues;
Enterobiasis, caused by infestation of the nematode Enterobius vermicularis, commonly called the human pinworm; the chief symptom is itching in the anal area;
Fascioliasis, a helminth disease caused by two trematodes Fasciola hepatica and Fasciola gigantica; symptoms may include fever, abdominal pain, loss of appetite, flatulence, nausea, diarrhea, urticarial, cough, dyspnoea, chest pain, hemoptysis, hepatomegaly, splenomegaly, ascites, anaemia, and jaundice in the acute phase, and inflammation and hyperplasia of the epithelium of the bile ducts in the chronic phase;
Fasciolopsiasis, caused by infection by the intestinal trematode Fasciolopsis buski, characterized by symptoms that may include abdominal pain, chronic diarrhea, anemia, ascites, toxemia, allergic responses, and intestinal obstruction;
Filariasis, an infectious tropical disease caused by thread-like roundworms belonging to the superfamily Filarioidea. Lymphatic filariasis is caused by the worms Wuchereria bancrofti, Brugia malayi, and Brugia timori, which occupy the lymphatic system, including the lymph nodes, and in chronic cases lead to the disease elephantiasis. Subcutaneous filariasis is caused by Loa loa (the eye worm), Mansonella streptocerca, and Onchocerca volvulus, which occupy the subcutaneous layer of the skin and Loa loa filariasis or river blindness. Serous cavity filariasis is caused by the worms Mansonella perstans and Mansonella ozzardi, which occupy the serous cavity of the abdomen.
Giardiasis, caused by the flagellate protozoan Giardia lamblia (also sometimes called Giardia intestinalis and Giardia duodenalis), characterized by symptoms that may include loss of appetite, diarrhea, hematuria (blood in urine), loose or watery stool, stomach cramps, upset stomach, projectile vomiting (uncommon), bloating, flatulence, and burping;
Gnathostomiasis, caused by the nematode Gnathostoma spinigerum and/or Gnathostoma hispidum; symptoms in the early stage of infection may include epigastric pain, fever, vomiting, and loss of appetite, and in later stages may include skin lesions that can be accompanied by pruritus, rash, and stabbing pain;
Hymenolepiasis, caused by infestation of the cestode Hymenolepis nana or Hymenolepis diminuta, characterized by symptoms that may include abdominal pain, loss of appetite (anorexia), itching around the anus, irritability and diarrhea;
Isosporiasis, a human intestinal disease caused by the parasite Isospora belli; symptoms may include acute, non-bloody diarrhea with crampy abdominal pain;
Katayama fever, caused by a parasitic worm of the genus Schistosoma; symptoms may range from mild anemia and malnutrition to abdominal pain, cough, diarrhea, eosinophilia, fever, fatigue, hepatosplenomegaly, and genital sores;
Leishmaniasis, caused by protozoan parasites of the genus Leishmania that are transmitted to humans by the bite of certain species of sand fly; symptoms may include skin sores which erupt weeks to months after the person affected is bitten by sand flies, and fever, damage to the spleen and liver, and anemia, which can manifest anywhere from a few months to years after infection;
Lyme disease, caused by at least three species of bacteria belonging to the genus Borrelia: Borrelia burgdorferi sensu stricto, Borrelia afzelii and Borrelia garinii. Early symptoms may include fever, headache, fatigue, depression, and a characteristic circular skin rash called erythema migrans (EM), while later symptoms may involve the joints, heart, and central nervous system;
Malaria, a mosquito-borne infectious disease of caused by protozoa of the genus Plasmodium, characterized by symptoms that may include fever, chills, headache, sweats, fatigue, anemia, nausea, dry (nonproductive) cough, muscle and/or back pain, an enlarged spleen, hemolytic anemia, kidney failure, liver failure, meningitis, pulmonary edema, and hemorrhaging from the spleen;
Metagonimiasis, caused by the intestinal trematodes Metagonimus yokagawai, M. takashii or M. miyatai; characterized by diarrhea, colicky abdominal pain, lethargy and/or anorexia;
Onchocerciasis, also known as river blindness and Robles disease, caused by the nematode Onchocerca volvulus and its endosymbiont Wolbachia pipientis; symptoms may include subcutaneous nodules in the early phase, and skin symptoms including intense itching, swelling, inflammation, papular onchodermatitis, skin atrophy, and depigmentation, and ocular symptoms including punctate or sclerosing keratitis of the cornea at later phases;
Scabies, a contagious skin infection caused by the mite Sarcoptes scabiei; characterized by intense allergic itching;
Schistosomiasis, a parasitic disease caused by several species of trematodes of the genus Schistosoma; symptoms may range from mild anemia, malnutrition, mild itching and a papular dermatitis to abdominal pain, cough, diarrhea, eosinophilia, fever, fatigue, hepatosplenomegaly, and genital sores;
Sleeping sickness (also known as African trypanosomiasis, African lethargy, or Congo trypanosomiasis) caused by protozoa of the species Trypanosoma brucei and transmitted by the tsetse fly; the first stage (the haemolymphatic phase) is characterized by one or more of fever, headaches, joint pains, itching, anemia, and endocrine, cardiac, and kidney disfunctions, while the second stage (the neurological phase) is characterized by confusion, reduced coordination, and disruption of the sleep cycle, with bouts of fatigue punctuated with manic periods, leading to daytime slumber and night-time insomnia;
Strongyloidiasis, caused by the nematode Strongyloides stercoralis, or sometimes S. fülleborni; symptoms may include skin symptoms (e.g. urticarial rashes in the buttocks and waist areas), abdominal pain, diarrhea and weight loss;
Taeniasis, caused by cestodes of the genus Taenia (e.g. T. solium, and T. saginata); it is generally asymptomatic and is diagnosed when a portion of the worm is passed in the stool.
Theileriosis, a malaria-like disease caused by a protozoan of the genus Theileria, e.g. in humans by T. microti; in horses, by T. equi (“Equine Piroplasmosis”); in sheep and goats, by T. lestoquardi; and in cattle, African buffalo, water buffalo, and water bucks, by T. annulata (“Tropical Theileriosis”) or T. parva (“East Coast fever”); symptoms include causes fever, hemolysis, anemia, jaundice, hypothermia, and heart failure.
Toxocariasis, caused by a larvae of either the dog roundworm (Toxocara canis), the cat roundworm (Toxocara cati) or the fox (Toxocara canis); it presents as one of three syndromes: visceral larva migrans (VLM), which is caused by high parasite load (symptoms include pallor, fatigue, weight loss, anorexia, fever, headache, rash, cough, asthma, chest tightness, increased irritability, abdominal pain, nausea, vomiting); covert toxocariasis, which is a milder version of VLM caused by chronic exposure to parasite (symptoms include coughing, fever, abdominal pain, headaches, and changes in behavior and ability to sleep); and ocular larva migrans (OLM), which is associated with a light Toxocara burden, and in which pathological effects on the host are restricted to the eye and the optic nerve;
Toxoplasmosis, caused by the protozoan Toxoplasma gondii; it is typically asymptomic, but in AIDS patients and pregnant women, may cause encephalitis (inflammation of the brain), neurologic diseases, and complications of the heart, liver, inner ears, and eyes (chorioretinitis);
Trichinosis, caused by the larvae of a species of roundworm Trichinella spiralis (T. spiralis, T. nativa, and T. britovi); infected individual may be asymptomatic, or may present with symptoms nausea, heartburn, dyspepsia, and diarrhea from two to seven days after infection, edema, muscle pain, fever, and weakness thereafter; Trichuriasis, caused by the intestinal parasitic nematode Trichuris trichiura; symptoms include as gastrointestinal problems including abdominal pain and distention, bloody or mucus-filled diarrhea, and tenesmus (feeling of incomplete defecation).
Nonlimiting examples of parasitic diseases in non-human animals (e.g., livestock, cattle, dogs, cats, sheep, goats, pigs, horses, lamas, etc.) that may be treated by the methods and compositions of the present invention include:
Anaplasmosis, formerly known as gall sickness, traditionally refers to a disease of ruminants caused by obligate intraerythrocytic bacteria of the order Rickettsiales, family Anaplasmataceae, genus Anaplasma. Cattle, sheep, goats, buffalo, and some wild ruminants can be infected with the erythrocytic Anaplasma. The Anaplasma genus also includes Anaplasma phagocytophilum (compiled from species previously known as Ehrlichia phagocytophila, E equi, and human granulocytic ehrlichiosis agent), A bovis (previously E bovis), and A platys (previously E platys), all of which invade blood cells other than erythrocytes of their respective mammalian hosts. Clinical bovine anaplasmosis is usually caused by A marginale. An A marginale with an appendage has been called A caudatum, but it is not considered to be a separate species. Cattle are also infected with A centrale, which generally results in mild disease. A ovis may cause mild to severe disease in sheep, deer, and goats. Up to 19 different tick vector species (including Boophilus, Dermacentor, Rhipicephalus, Ixodes, Hyalomma, and Ornithodoros) have been reported to transmit Anaplasma spp. Anaplasmosis is characterized by progressive anemia due to extravascular destruction of infected and uninfected erythrocytes. Animals with peracute infections succumb within a few hours of the onset of clinical signs, which can include fallen milk production, inappetence, loss of coordination, breathlessness when exerted, and a rapid bounding pulse, and brown urine;
Babesiosis, caused by intraerythrocytic protozoan parasites of the genus Babesia, is transmitted by ticks, affects a wide range of domestic and wild animals and occasionally people. The two most important species in cattle are B bigemina and B bovis. The main vectors of Babesia bigemina and B bovis are 1-host Rhipicephalus (Boophilus) spp ticks, in which transmission occurs transovarially. Symptoms include fever, which persists throughout, inappetence, increased respiratory rate, muscle tremors, anemia, jaundice, and weight loss; hemoglobinemia and hemoglobinuria occur in the final stages. CNS involvement due to adhesion of parasitized erythrocytes in brain capillaries can occur with B bovis infections. Either constipation or diarrhea may be present. Cows: B divergens is transmitted by Ixodes ricinus, and B major by Haemaphysalis punctata. Equine babesiosis is caused by Theileria (formerly Babesia) equi or B caballi. Sheep and Goats: the 2 most important species are B ovis and B motasi. Ticks of the genera Rhipicephalus, Haemaphysalis, Hyalomma, Dermacentor, and Ixodes can be vectors. Pigs: Babesia trautmanni and B perroncitoi. Dogs and Cats: Babesia canis, B canis canis, B canis vogeli, and B canis rossi. Transmitted by Dermacentor reticularis, Rhipicephalus sanguineus, and Haemaphysalis leachi.
Cytauxzoonosis, caused by Cytauxzoon fells, is an infectious disease in domestic cats. Cytauxzoon spp are protozoan parasites classified within the family Theileriidae, along with Theileria spp and Gonderia spp. The bobcat (Lynx rufus) is the natural host, typically experiencing subclinical infection and maintaining chronic parasitemia. C felis infection has been reported in several other wild felids, such as cougars and panthers, in the absence of overt disease; however, a few lions and tigers have been reported to succumb to illness. Recent studies demonstrated that C felis can be transmitted by the lone star tick, Amblyomma americanum. Nonspecific signs include depression, lethargy, and anorexia. Fever and dehydration are the most common findings on a physical examination; body temperature rises gradually and can reach as high as 106° F. (41° C.). Other findings include icterus, lymphadenomegaly, and hepatosplenomegaly. In extremis, cats are often hypothermic, dyspneic, and vocalize as if in pain. Without treatment, death typically occurs within 2-3 days after peak in temperature;
Hemotropic Mycoplasmas (Hemoplasmas), which infect a wide variety of vertebrates throughout the world, share similar characteristics and morphologic features. They are pleomorphic, gram-negative bacteria lacking a cell wall and have not been cultured outside their hosts. Hemoplasmas attach to the surface of erythrocytes but do not penetrate the cell. Eperythrocytic parasites previously known as Haemobartonella and Eperythrozoon and formerly classified as rickettsial organisms have been reclassified as most closely related to members of the genus Mycoplasma. These organisms vary in their ability to cause clinically significant hemolytic anemia, but infected animals remain carriers despite antibiotic therapy. Parasitemia may reemerge if the animal is stressed or immunocompromised. Dogs: Mycoplasma haemocanis (formerly Haemobartonella canis) ‘Candidatus Mycoplasma haematoparvum’; Cats: Mycoplasma haemofelis (formerly Haemobartonella felis) ‘Candidatus Mycoplasma haemominutum’ ‘Candidatus Mycoplasma turicensis’; Pigs: Mycoplasma (Eperythrozoon) suis Eperythrozoon parvum (yet to be renamed); Cattle: Mycoplasma (Eperythrozoon) wenyonii; Sheep and goats: Mycoplasma (Eperythrozoon) ovis; and Llamas and alpacas: ‘ Candidatus Mycoplasma haemolamae’. Hemoplasmas are capable of causing a hemolytic anemia, but the severity varies greatly. The main exception is M haemofelis, which causes acute hemolytic anemia in healthy cats. The anemia may be severe and occasionally fatal. Typical clinical signs include lethargy, anorexia, and fever, with splenomegaly and icterus occurring less often. M haemocanis causes acute hemolysis in dogs that are splenectomized, but infections are usually asymptomatic in healthy dogs. M suis causes hemolytic anemia accompanied by icterus in neonatal pigs, feeder pigs, and pregnant sows. Chronic infection is associated with poor growth rates, decreased conception rates, reproductive failure, and decreased milk production. M wenyonii infection in cattle is usually asymptomatic, but a syndrome of mammary gland and hindlimb edema, decreased milk production, fever, and lymphadenopathy has been described in young primiparous heifers that were not anemic. M ovis infection in sheep and goats is often asymptomatic, but hemolytic anemia can occur in young animals, especially those with heavy intestinal worm burdens. Chronic infection may result in poor weight gain, exercise intolerance, decreased wool production, and mild anemia. Hemoplasma infection in camelids can cause a severe hemolytic anemia in young crias;
Hepatozoonosis (Old World and American Canine), a tickborne disease of wild and domestic carnivores, caused by the protozoal agent Hepatozoon canis. This organism is transmitted by the brown dog tick, Rhipicephalus sanguineus. The disease in North America is caused by H americanum, which is transmitted by the Gulf Coast tick, Amblyomma maculatum, rather than by the brown dog tick. Accordingly, the disease in North America is now recognized as a separate entity, American canine hepatozoonosis (ACH). Symptoms may include fever, depression, weight loss, poor body condition, muscle atrophy, soreness, stiffness, and weakness, mucopurulent ocular discharge is common, and bloody diarrhea occurs occasionally;
Schistosomiasis, a common parasitic infection in cattle and rarely in other domestic animals, is caused by schistosomes, which are members of the genus Schistosoma, family Schistosomatidae. Of the 19 species reported to naturally infect animals, 7—all parasites of ruminants—have received particular attention, mainly because of their recognized veterinary significance: S mattheei, S bovis, S curassoni, S spindale, S indicum, S nasale, and S japonicum. In the great majority of cases, visceral schistosome infections in endemic areas are subclinical and characterized by a high prevalence of low to moderate worm burdens in the cattle population. Although few or no overt clinical signs may be recognized in the short term, high prevalence rates of chronic schistosome infections cause significant losses on a herd basis.
Theileriases, a group of tickborne diseases caused by Theileria spp. A large number of Theileria spp are found in domestic and wild animals in tick-infested areas of the Old World. The most important species affecting cattle are T parva (which causes “East Coast fever”, also known as “Corridor disease”) and T annulata (which causes “Tropical Theileriosis”, also known as “Mediterranean theileriosis”), both of which cause widespread death in tropical and subtropical areas of the Old World. T lestoquardi, T lowenshuni, and T uilenbergi are important causes of mortality in sheep. Theileria use, successively, WBC and RBC for completion of their life cycle in mammalian hosts. Typically, fever occurs 7-10 days after parasites are introduced by feeding ticks, continues throughout the course of infection, and may be >107° F. (42° C.). Lymph node swelling becomes pronounced and generalized. Lymphoblasts in Giemsa-stained lymph node biopsy smears contain multinuclear schizonts. Anorexia develops and the animal rapidly loses condition; lacrimation and nasal discharge may occur. Terminally, dyspnea is common. Just before death, a sharp fall in body temperature is usual, and pulmonary exudate pours from the nostrils.
Trypanosomiasis, a group of diseases caused by protozoa of the genus Trypanosoma affects all domestic animals. The major species are T congolense (Cattle, sheep, goats, dogs, pigs, camels, horses, most wild animals), T vivax (Cattle, sheep, goats, camels, horses, various wild animals), T brucei brucei (All domestic and various wild animals; most severe in dogs, horses, cats), and T simiae (Domestic and wild pigs, camels). Severity of disease varies with species and age of the animal infected and the species of trypanosome involved. The primary clinical signs are intermittent fever, anemia, and weight loss. Cattle usually have a chronic course with high mortality, especially if there is poor nutrition or other stress factors. Ruminants may gradually recover if the number of infected tsetse flies is low; however, stress results in relapse.

One parasitic disease in humans of particular interest is malaria. Malaria is a mosquito-borne infectious disease of humans and other animals caused by protozoa of the genus Plasmodium. It begins with a bite from an infected Anopheles mosquito, which introduces the protozoa via its saliva into the circulatory system, and ultimately to the liver where they mature and reproduce. The parasites then enter the bloodstream and infect red blood cells. The disease causes symptoms that typically include fever and headache, which in severe cases can progress to coma or death.

Malaria is widespread in tropical and subtropical regions in a broad band around the equator, including much of Sub-Saharan Africa, Asia, and the Americas. Malaria is prevalent in these tropical and subtropical regions because rainfall, warm temperatures, and stagnant waters provide habitats ideal for mosquito larvae.

Five species of Plasmodium can infect and be transmitted by humans. The vast majority of deaths are caused by P. falciparum, while P. vivax, P. ovale, and P. malariae cause a generally milder form of malaria that is rarely fatal. The zoonotic species P. knowlesi, prevalent in Southeast Asia, causes malaria in macaques but can also cause severe infections in humans. P. falciparum causes severe malaria via a distinctive property not shared by any other human malaria, that of sequestration. Within the 48-hour asexual blood stage cycle, the mature forms change the surface properties of infected red blood cells, causing them to stick to blood vessels (a process called cytoadherence). This leads to obstruction of the microcirculation and results in dysfunction of multiple organs.

Symptoms of malaria include fever, chills, headache, sweats, fatigue, anemia, nausea, dry (nonproductive) cough, muscle and/or back pain, and an enlarged spleen. Other symptoms and complications associated with malaria include brain infection (cerebritis), hemolytic anemia, kidney failure, liver failure, meningitis, pulmonary edema, and hemorrhaging from the spleen. In other words, an individual having malaria may display one or more of fever, chills, headache, sweats, fatigue, nausea, dry (nonproductive cough), muscle and/or back pain, and an enlarged spleen, and in more severe cases may demonstrate brain infection (cerebritis), hemolytic anemia, kidney failure, liver failure, meningitis, pulmonary edema, or hemorrhaging from the spleen. An individual at risk for developing malaria is at risk for, but has not yet developed, symptoms of malaria including fever, chills, headache, sweats, fatigue, nausea, dry (nonproductive cough), muscle and/or back pain, or an enlarged spleen. Generally, an individual at risk for developing malaria will begin to show symptoms 7 days or more after infection, e.g., 9 to 14 days after the initial infection by P. falciparum, 12 to 18 days after the initial infection by P. vivax or P. ovale, 18 to 40 days after the initial infection by P. malariae, or 11 to 12 days after the initial infection by P. knowlesi.

Anti-malaria agents used in the art to treat or prevent malaria include chloroquine, quinidine, doxycycline, tetracycline, clindamycin, atovaquone plus proguanil (Malarone), Mefloquine, artesunate, and pyrimethamine plus sulfadoxine (Fansidar).

Another parasitic disease of particular interest is Babesiosis, a malaria-like parasitic disease caused by infection with the protozoa Babesia. Babesiosis is a vector-borne illness usually transmitted by Ixodes scapularis ticks. The disease is typically caused by B. microti in humans, B. canis rossi and B. canis canis in dogs, B. bovis in cows, and B. bigemina in cattle. Babesia microti, which infects humans, uses the same tick vector as Lyme disease and ehrlichiosis, and may occur in conjunction with these other diseases. The protozoa can also be transmitted by blood transfusion.

In humans, babesiosis may be asymptomatic, or characterized by symptoms ranging from mild fever and diarrhea to high fever, shaking chills, and severe anemia. In other words, an individual having babesiosis may display one or more of fever, diarrhea, shaking chills, and anemia. In severe cases, organ failure, including respiratory distress syndrome, may occur. Severe cases occur mostly in people who have had a splenectomy, or persons with an immunodeficiency, such as HIV/AIDS patients. In animals, B. canis rossi, B. bigemina, and B. bovis cause particularly severe forms of the disease, including a severe haemolytic anaemia. Common sequelae include haemoglobinuria “red-water”, disseminated intravascular coaguation and “cerebral babesiosis” caused by sludging of erythrocytes in cerebral capillaries. Infected animal will show pale mucous membranes initially, due to the hemolytic anemia. As the levels of billirubin (a byproduct of red blood cell lysis) continue to increase, the visible mucous membranes will become yellow in color (icterus) due to the failure of the liver to metabolise the excess bilirubin. Hemoglobinuria will be seen due to excretion of red-blood-cell lysis byproducts via the kidneys. Fever of 40.5° C. (105° F.) develops due to release of inflammatory byproducts.

Definitive diagnosis of infection by Babesia is by the identification of the parasite on a Giemsa-stained thin blood smear. The parasite appears in erythrocytes as paired merozoites forming the “Maltese cross formation” in humans or “two pears hanging together” in animals. Other diagnostic methods include PCR of peripheral blood, and serologic testing for antibodies (IgG, IgM) against Babesia.

Most cases of babesiosis in humans resolve without any specific treatment. Treatment, when provided, typically comprises a two-drug regimen of quinine and clindamycin, or of atovaquone and azithromycin. In instances where babesiosis appears life-threatening, a blood exchange transfusion is performed, in which infected red blood cells are removed and replaced with uninfected ones. In animals, treatment of babesiosis typically involves the administration of diminazen (Berenil), imidocarb or trypan blue.

Another parasitic disease of particular interest is Theileriosis. Theileriosis is a malaria-like disease caused by a protozoan of the genus Theileria. For example, in humans, theileriosis may be caused by T. microtia in horses, by T. equi (“Equine Piroplasmosis”); in sheep and goats, by T. lestoquardi; and in cattle, African buffalo, water buffalo, and water bucks, by T. annulata (“Tropical Theileriosis”, also known as “Mediterranean theileriosis”) or T. parva (“East Coast fever”, also known as “Corridor disease”). Theirleriosis is transmitted to the host by various tick species including Ixodes scapularis, Rhipicephalus, Dermacentor, Haemaphysalis, and Hyalomma. The organism reproduces in the tick as it progresses through its life stage, and matures and enters the saliva after the tick attaches to a host. Usually, the tick must be attached for a few days before it becomes infective. However, if environmental temperatures are high, infective sporozoites can develop in ticks on the ground, and may enter the host within hours of attachment.

Theirleriosis in humans typically presents as fever and hemolysis. In animals, East Coast fever, caused by T. parva and common in Eastern and Southern Africa, usually manifests 7-10 days after infection, and typically presents with lymphadenopathy, fever, anorexia and loss of condition with decreased milk yield. Petechiae and ecchymoses may be found on the conjunctiva and oral mucous membranes. Lacrimation, nasal discharge, corneal opacity and diarrhea can also be seen. Terminally ill animals often develop pulmonary edema, severe dyspnea and a frothy nasal discharge. Some cattle have a fatal condition called “turning sickness.” In this form of the disease, infected cells block capillaries in the central nervous system and cause neurological signs. Tropical theileriosis, caused by T. annulata and common in North Africa, southern Europe, and Asia, generally resembles East Coast fever, but these parasites also destroy red blood cells, causing jaundice, anemia, and in some cases, hemoglobinuria. Hemorrhagic diarrhea may be seen in the late stages. Petechiae are often found on the mucous membranes. Neurological signs have been documented in some terminally ill water buffalo, but “turning sickness” does not seem to be a feature of tropical theileriosis in cattle. Theileriosis in small ruminants, caused by T. lestoquardi and common in the Mediterranean, North Africa, and Asia, presents with fever, anorexia and weight loss, listlessness, lymphadenopathy, edema of the throat, difficulty breathing, anemia and icterus. Subacute, chronic or mild cases can also be seen.

Definitive diagnosis of infection by Theileria is by the identification of the parasite on a Giemsa-stained thin blood smear. Theileriosis in humans typically resolves without any specific treatment. Treatment in animals typically includes administration of halofuginone, oxytetracycline, primaquine or buparvaquone.

In some instances, the parasitic disease is associated with a parasite having a genome that comprises one or more ALDH genes. In other words, the subject is infected with or at risk of becoming infected with a parasite having a genome that comprises one or more ALDH genes. Non-limiting examples of such parasites include Mycobacterium tuberculosis, Toxoplasma gondii, Leishmania braziliensis, and Trypanosoma cruzi. Such parasites may be readily identified using any convenient technique for determining if an organism carries a gene of interest, e.g. DNA sequence analysis of the genome of the organism, enzymatic analysis of a lysate prepared from the organism to detect gene activity, etc. as known in the art or described below.

In other instances, the parasitic disease is associated with a parasite having a genome that does not comprise any ALDH genes. In other words, the subject is infected with or at risk of becoming infected with a parasite having a genome that comprises no ALDH genes. Non-limiting examples of parasites that do not comprise an ALDH gene in their genome include protozoa that are hematozoa, e.g. protozoa of the Plasmodium family, e.g. P. falciparum, P. vivax, P. ovale, P. malariae and P. knowlesi; protozoa of the Babesia genus, e.g. B. bigemina, B. bovis, B. canis, B. cati, B. divergens, B. duncani, B. felis, B. gibsoni, B. herpailuri, B. jakimovi, B. major, B. microti, B. ovate, B. pantherae; and protozoa of the Theileria genus, e.g. T. annulata, T. electrophori, T. equi, T. lestoquardi, T. microti, T. orientalis, and T. parva. Such parasites may be readily identified using any convenient technique for determining if an organism carries a gene of interest, e.g. DNA sequence analysis of the genome of the organism, enzymatic analysis of a lysate prepared from the organism to detect gene activity, etc. as known in the art or described below.

### Reagents, Devices and Kits

Also provided are reagents, devices and kits thereof for practicing one or more of the above-described methods. The subject reagents, devices and kits thereof may vary greatly. For example, kits may comprise one or more ALDH antagonists as described above or known in the art. Kits may also comprise one or more additional anti-parasitic agents, e.g. acetaldehyde, 4HNE, or another antiparasitic agent as described above or known in the art. In some instants, the kit may comprise one or more reagents or devices useful for detecting an infection by a parasite, for example, blood films for detection of the parasite microscopically in blood; parasite antigen-specific dye-labeled antibody, lysis buffer, and/or test strips for detection of the parasite in a fluid or tissue sample by, e.g., immunochromatography; primers and/or PCR reagents for detection of the parasite in a fluid or tissue sample by PCR, etc. In some instants, the kit may comprise one or more reagents and devices useful for detecting the symptoms of the parasitic disease, e.g. a thermometer calibrated to detect fever in a human, blood pressure monitor, blood collection vessel such as a collection tube or capillary pipette, reagents to perform a complete blood count, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

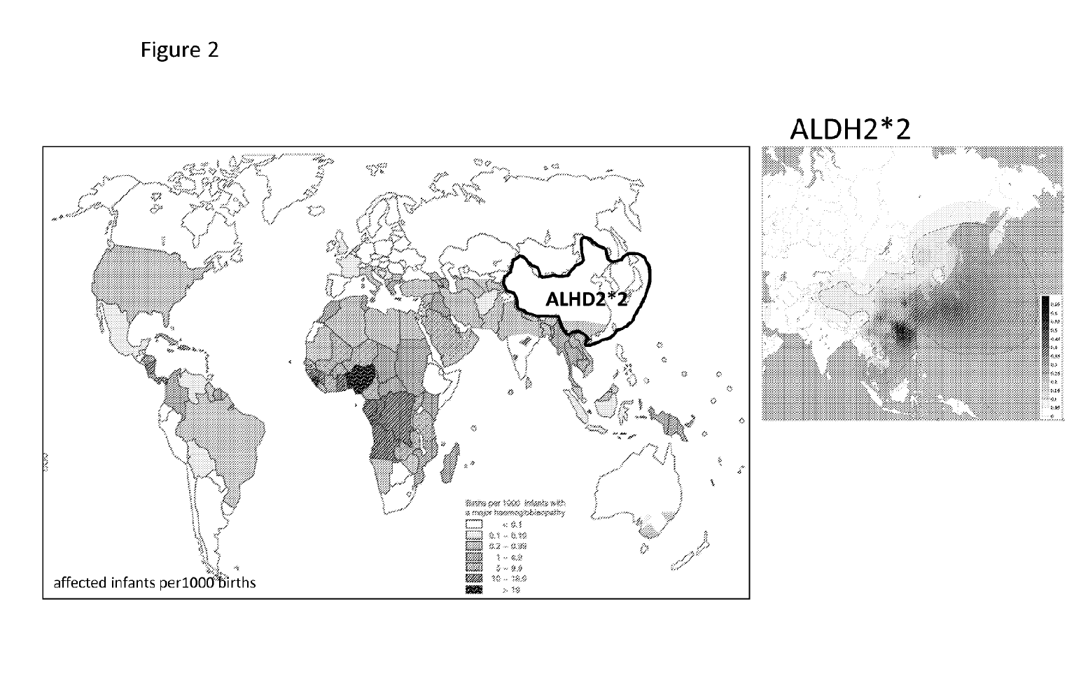

Several genetic mutations have evolved to confer natural resistance against malaria parasite infection. These loss-of-function mutations underlie sickle cell anemia, thalassemia, and glucose-6-phosphate dehydrogenase (G6PD) deficiency, with each of these affecting ˜200-400 million people concentrated in malaria-infected areas. Large scale global genetic maps indicate that the distribution of these red blood cells (RBC) defects occupy well defined, separate, and non-overlapping geographic regions relative to the region of ALDH2*2 prevalence (Peters, A. L. et al. (2009) Glucose-6-phosphate dehydrogenase deficiency and malaria: cytochemical detection of heterozygous G6PD deficiency in women. J Histochem Cytochem, 57(11): p. 1003-11; Li H., et al. (2009) Refined geographic distribution of the oriental ALDH2*504Lys (nee 487Lys) variant. Ann Hum Genet. 73 (Pt 3):335-45; WHO, “Global distribution of haemoglobin disorders”, found on the world wide web at http:// followed by “www.who.int/genomics/public/Maphaemoglobin.pdf_”) (FIG. 2). Specifically, high frequencies of sickle cell anemia, thalassemia and G6PD deficiency are found in malaria-endemic Africa, South America, India and South Asia (FIG. 2). In contrast, in East Asia, where malaria has historically been equally rampant, relatively low rates of these mutations are found, but nearly 40% of the population in East Asia carries the E487K amino acid substitution in the gene encoding ALDH2 (Eng, M. Y., et al. ALDH2, ADH1B, and ADH1C genotypes in Asians: a literature review. Alcohol Res Health, 2007; 30(1): p. 22-7; Brooks P. J., et al. The alcohol flushing response: an unrecognized risk factor for esophageal cancer from alcohol consumption. PLoS Med. 2009; 6(3):e50).

The ALDH2*2 mutation is a loss-of-function enzyme deficiency in humans. ALDH2 is a critical detoxifying enzyme that catalyzes conversion of acetaldehyde and other oxidative stress-derived reactive aldehydes to their non-toxic acids. Two key enzymes, alcohol dehydrogenase (ADH) and aldehyde dehydrogenase (ALDH) are required for ethanol metabolism (Zakhari S., et al. Determinants of alcohol use and abuse: Impact of quantity and frequency patterns on liver disease. Hepatology. 2007; 46(6):2032-9). Ethanol is first oxidized by ADH to acetaldehyde, which is then further oxidized by ALDH to non-toxic acetate. Among the 19 known human ALDH isozymes, mitochondrial ALDH2 is the most efficient catalyst for conversion of ethanol-derived toxic acetaldehyde at physiologically relevant concentrations. As illustrated in FIG. 2, the ALDH2*2 mutation affects ˜560 million people (8% of the world's population) and originated in malaria-afflicted South East China ˜3000 years ago (Brooks P. J., et al. (2009) The alcohol flushing response: an unrecognized risk factor for esophageal cancer from alcohol consumption. PLoS Med. 6(3):e50; Luo H. R., et al. (2009) Origin and dispersal of atypical aldehyde dehydrogenase ALDH2 487Lys. Gene 435(1-2):96-103). Carriers of this mutation have drastically reduced enzyme capacity for aldehyde detoxification (˜16-40% in heterozygous, ˜1-5% in homozygotes individuals) (Wang X., et al. (1996) Heterotetramers of human liver mitochondrial (class 2) aldehyde dehydrogenase expressed in Escherichia coli. A model to study the heterotetramers expected to be found in Oriental people. J Biol Chem. 271(49):31172-8; Chen C. H., et al. (2008) Activation of aldehyde dehydrogenase-2 reduces ischemic damage to the heart. Science 321(5895):1493-5). The ALDH2*2 mutation is associated with elevated acetaldehyde in the liver and in circulating blood following even a very moderate alcohol consumption (Chen Y. C., et al. (2009) Pharmacokinetic and pharmacodynamic basis for overcoming acetaldehyde-induced adverse reaction in Asian alcoholics, heterozygous for the variant ALDH2*2 gene allele. Pharmacogenet Genomics 19(8):588-99) and manifest a well-characterized “ethanol-induced Asian Flushing” due to the acetaldehyde accumulation.

Despite the prevalence of ALDH2*2, no obvious explanation or known selective advantage for this common enzyme deficiency has to date been found (Lin Y. P., et al. (2002) Why can't Chinese Han drink alcohol? Hepatitis B virus infection and the evolution of acetaldehyde dehydrogenase deficiency. Med Hypotheses 59(2):204-7). Our data indicates that ALDH2*2-dependent enzyme deficiency is maintained in human populations because it confers a natural resistance to malaria parasite infection through the accumulation of aldehydes that are toxic to malaria parasites. As such, ALDH2 provides a new anti-malarial drug target for both liver and RBC-stage parasites. New preventative and therapeutic ALDH-based anti-parasitic strategies will render parasites susceptible to aldehyde toxicity.

### Results

ALDH is highly conserved and essential for detoxification of aldehydes in all living organisms ranging from bacteria to mammals (Sophos N. A., et al. (2003) Aldehyde dehydrogenase gene superfamily: the 2002 update. Chem Biol Interact. 143-144:5-22; Marchitti S. A., et al. (2008) Non-P450 aldehyde oxidizing enzymes: the aldehyde dehydrogenase superfamily. Expert Opin Drug Metab Toxicol. 4(6):697-720). The presence of ALDH encoding genes in the vast majority of archaeal, eubacterial and eukaryotic genomes supports the notion that these enzymes are important components of metabolic processes in living organisms and that the ALDH superfamily is ancient in origin. Complete sequencing of individual genomes reveals the number of ALDH genes found per organism ranges from 1 to 5 in archaeal species, 1 to 26 genes in eubacterial species, and 8 to 17 genes in eukaryotic species (Sophos N. A., et al. Aldehyde dehydrogenase gene superfamily: the 2002 update). Our own searches for ALDH-encoding genes by key word queries and DNA sequence analyses of 19 organisms/species ranging from single-celled eukaryotes, including several human parasites, to a variety of metazoans, demonstrated that 16 of 19 species had at least one copy of ALDH in their genomes. The number of ALDH genes was in general positively associated with organism complexity, with fewer copies in the simpler life forms (bacteria, protists) and multiple copies of encoded ALDH in more highly evolved organisms such as zebrafish (18 copies) and humans (19 copies) (Table 1). The genome of the kinetoplastid parasites Leishmania braziliensis encodes 4 ALDH genes and that of T. cruzi encodes 5 ALDH genes. The genome of Toxoplasma gondii, a close phylogenetic relative of Plasmodium spp., encodes 5 ALDH genes. However, we could identify no ortholog of ALDH within the genomes of any species of Plasmodium, including all known human, non-human primate and rodent Plasmodium parasites, nor in the genomes of Babesia bovis or Theileria parva. These observations suggest that ALDH activity is critical for the survival of organisms, and that parasites of the genera Plasmodium, Babesia and Theileria have evolved to be strictly dependent on host ALDH function for toxic aldehyde removal. Up to now, there has been no precedence or report on the lack of ALDH gene in any of the living organisms.

TABLE 1
Survey of ALDH gene copy number in 19 different organisms. Results are based
on key word searches of protein, gene names and DNA, protein sequence homology blasts
of all available public genome databases.

| | Genome size | No. of | No. of |
|---|---|---|---|
| Organism | (million base pair) | Genes | ALDH Genes |
| Escherichia coli | 4.6 | 4,377 | 8 |
| Mycobacterium tuberculosis | 4.4 | 3,959 | 7 |
| Saccharomyces cerevisiae | 12.4 | 5,770 | 7 |
| Cyanidioschyzon merolae | 16.5 | 5,331 | 6 |
| Plasmodium falciparum | 22.8 | 5,268 | 0 |
| Plasmodium vivax | ~27.0 | 5,400 | 0 |
| Plasmodium knowlesi | 23.5 | 5,118 | 0 |
| Plasmodium yoelii | 23.1 | 5,878 | 0 |
| Babesia bovis | 8.2 | 3,671 | 0 |
| Theileria parva | 8.3 | 4,035 | 0 |
| Toxoplasma gondii | ~70 | ~8,000 | 5 |
| Leishmania braziliensis | 32.8 | 8,272 | 4 |
| Trypanosoma cruzi | 35.0 | 12,570 | 5 |
| Anopheles gambiae (host) | 278 | 14,000 | >7 |
| Caenorhabditis elegans | 100 | 21,733 | 13 |
| Arabidopsis thaliana | 115 | 28,000 | 14 |
| Drosophila melanogaster | 122 | 17,000 | 14 |
| Danio rerio | 1,200 | 15,761 | 18 |
| Human (host) | 3,300 | 21,000 | 19 |

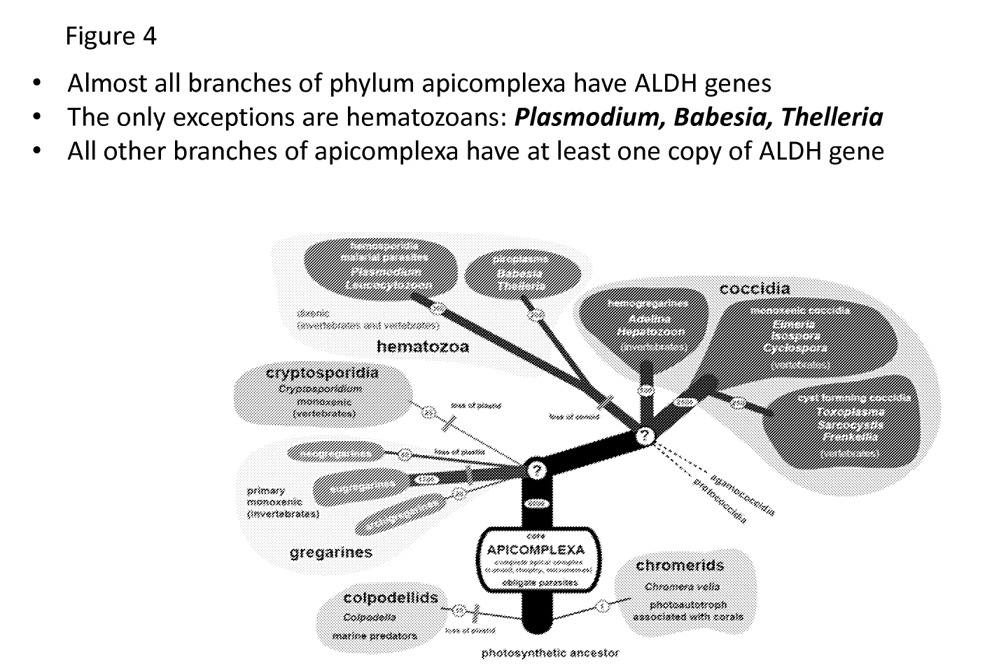

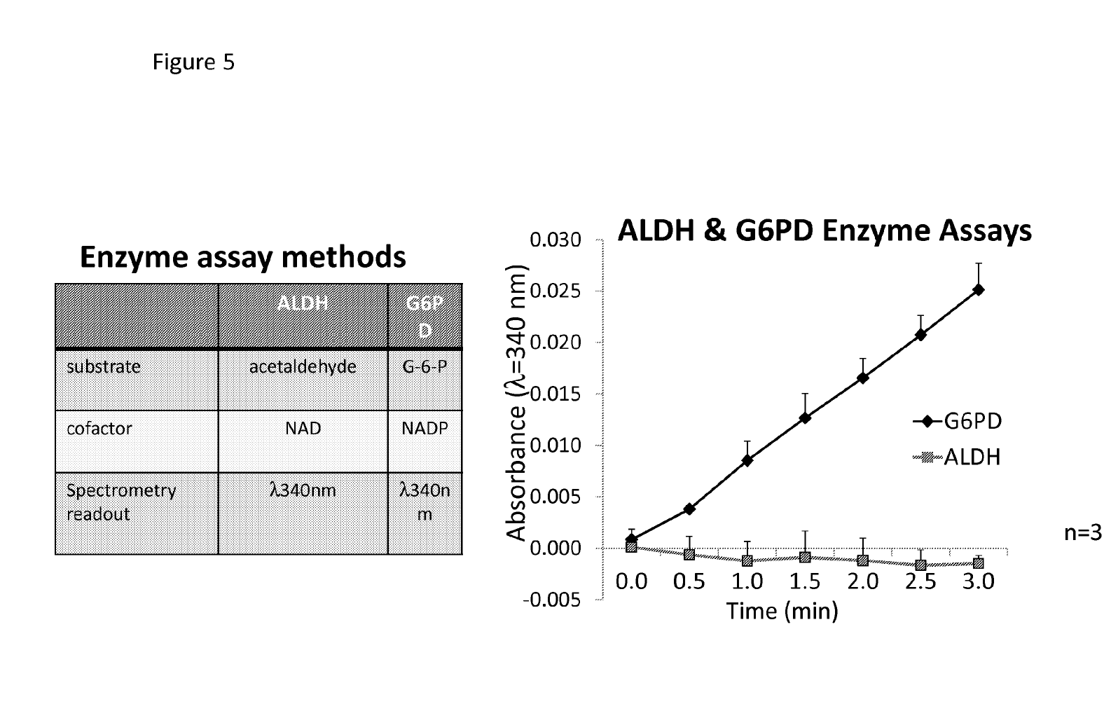

As illustrated in FIG. 4, Plasmodium, Babesia and Theileria are closely related based on the taxonomy of apicomplexa. Parasites from all 3 genera infect erythrocytes and belong to hematozoa. It is likely that ALDH gene(s) are lost in the common ancestor of the hematozoa. We carried out an enzymatic assay for ALDH activity in lysates from cultured P. falciparum. We found no measurable ALDH enzymatic activity in parasite lysates. As a positive control, the enzymatic activity of a known parasite dehydrogenase, G6PD, was easily detected in our assay (FIG. 5). The lack of encoded ALDH function in P. falciparum was a surprising and novel finding that suggested that malaria parasites may be incapable of detoxifying aldehydes and that these compounds could be exploited as parasiticides. Acetaldehydes and 4HNE are toxic to malaria parasites in vitro (Becker K., et al. Antimalarial activity of the ethanol/alcohol oxidase system in vitro. Free Radic Res Commun. 1990; 9(1):33-8; Clark I. A., et al. Toxicity of certain products of lipid peroxidation to the human malaria parasite Plasmodium falciparum. Biochem Pharmacol. 1987; 36(4):543-6); 50-100 μM 4HNE reduced growth of P. falciparum by 50-100% relative to controls (Clark I. A., et al. Toxicity of certain products of lipid peroxidation to the human malaria parasite Plasmodium falciparum. Biochem Pharmacol. 1987; 36(4):543-6).

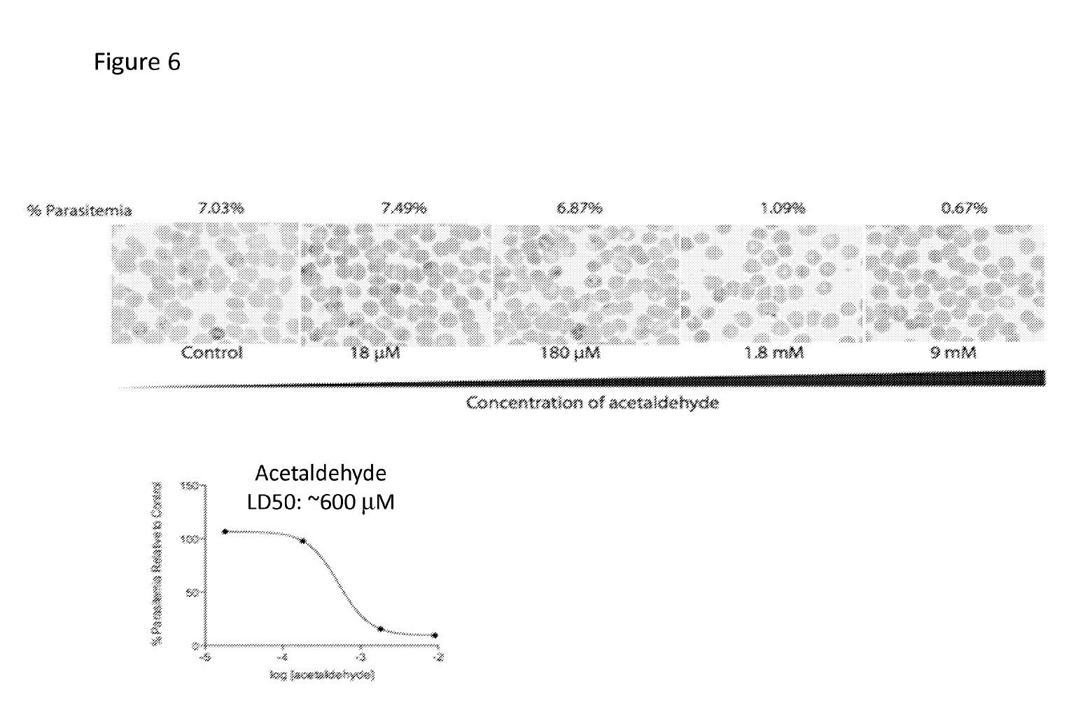

Our preliminary data demonstrate that acetaldehyde (μM range) can also inhibit growth of P. falciparum in human RBCs (FIG. 6). In these studies, a day 10 P. falciparum culture is treated with acetaldehyde, the culture is incubated for 28 h, the parasites are quantified by smear count by Geimsa stain and flow cytometry, and parasitemia is determined as percent of red blood cells stained positively with the presence of parasites. These growth inhibiting concentrations of acetaldehyde are clinically relevant. Specifically, in published data from human ALDH2*2 heterozygotic subjects, consumption of merely 2.5 alcoholic drinks (0.5 g/kg body weight), leads to 60 μM blood acetaldehyde levels, as compared with ˜2 μM in subjects with wild type enzyme (Chen Y. C., et al. Pharmacokinetic and pharmacodynamic basis for overcoming acetaldehyde-induced adverse reaction in Asian alcoholics, heterozygous for the variant ALDH2*2 gene allele. Pharmacogenet Genomics. 2009; 19(8):588-99). In the liver and hepatocytes of these subjects, where 90% of the alcohol is metabolized, acetaldehyde concentration is expected to be much higher than in blood. Acetaldehyde concentration, at 450 μM or higher, has been detected in human saliva after drinking alcohol (Homann et al., 1997; Carcinogenesis, 18:1739-1743, Visapaa et al., 2004; Gut, 53: 871-876). In the colon of rats following ethanol treatment, 2.7 mM has been detected (Visapaa et al., 1998; Alcohol Clin. Exp. Res., 22: 1161-1164). It is expected that in the hepatocytes of ALDH2*2 human individuals low mM concentrations of acetaldehyde exists under physiological condition. Indeed, a 10-fold increase of acetaldehyde adducts have been detected in ALDH2^{-/-} mice after drinking a liquid ethanol diet (Nagayoshi et al., 2009; Mutat Res., 673: 74-77). Further, μM concentrations of 4HNE are also very common under conditions of infection, inflammation or fever (e.g. during the chill-fever cycles of malaria). Therefore, physiologically relevant concentrations of aldehydes may inhibit growth of P. falciparum.

### DISCUSSION

The complete P. falciparum genome was published 10 years ago (Carlton J. M., et al. Genome sequence and comparative analysis of the model rodent malaria parasite Plasmodium yoelii yoelii. Nature. 2002; 419(6906):512-9), followed by the genomes of a variety of other Plasmodium species (Bahl A., et al. PlasmoDB: the Plasmodium genome resource. An integrated database providing tools for accessing, analyzing and mapping expression and sequence data (both finished and unfinished). Nucleic Acids Res. 2002; 30(1):87-90). To our knowledge, however, we are the first to report the absence of encoded ALDH orthologs in all available Plasmodium genomes. The lack of recognizable ALDH orthologs by blast search of PlasmoDB is supported by a lack of detectable ALDH activity in cultured P. falciparum (FIG. 5). This lack of ALDH activity indicates that malaria parasites lack the ability to detoxify aldehydes, a feature of malaria parasites that could be exploited to develop new anti-malarial strategies.

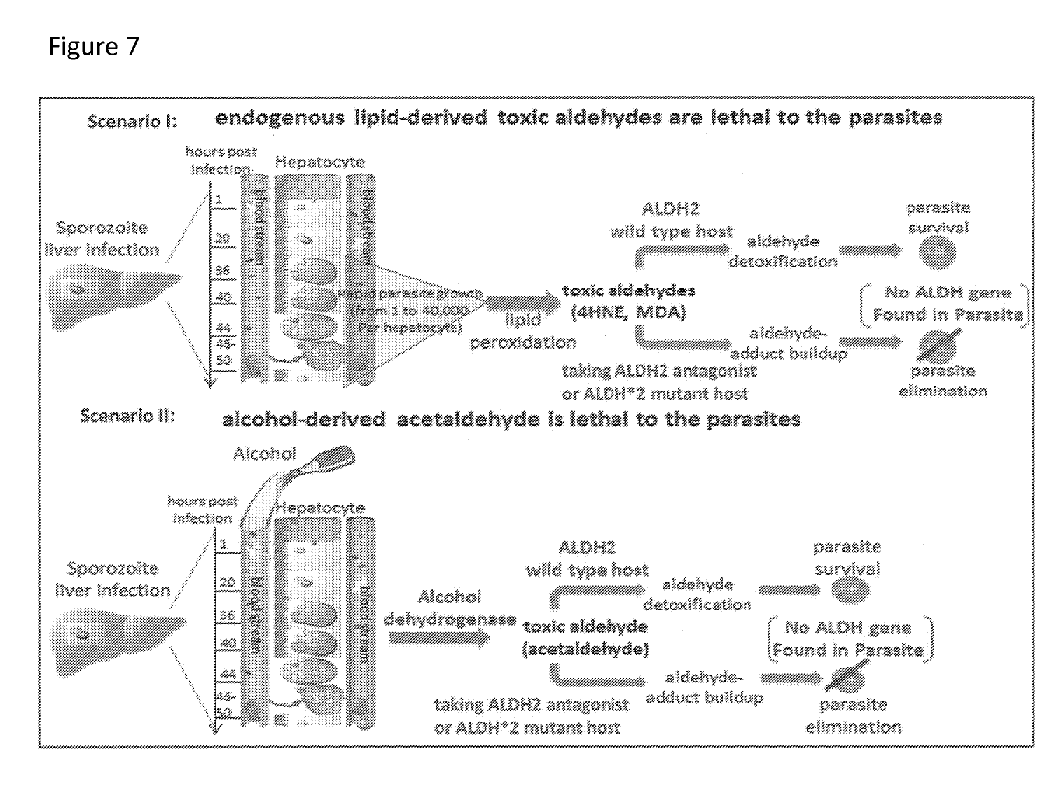

Drugs that target liver stage malaria parasites are rare due to the lack of specific liver targets. Since ALDH2 is most abundantly expressed in the liver, suppression of ALDH2 function (or as occurring naturally as the ALDH2*2 mutation in East Asians) in particular in this organ could be an ideal strategy for the development of novel anti-liver stage drugs. As illustrated in FIG. 7, two likely source of toxic aldehydes could be generated to explain the selective advantage of ALDH2*2 against malaria. In the first scenario, endogenous lipid-derived aldehydes, 4HNE and MDA, are accumulated as toxic byproducts during the rapid proliferation and growth phase of the parasites in hepatocytes. In the second scenario, elevated toxic acetaldehyde is accumulated after alcohol consumption, or other exogenous sources. Targeting ALDH2 will not only adversely impact liver stage parasites, but also blood stage parasites due high level of circulating toxic aldehydes. The lack of identifiable encoded ALDH orthologs in any malaria parasite species suggests that this strategy would also be useful against infections with any malaria parasite species. Targeting ALDH2 will, therefore, be effective in interrupting all stages of the parasite life cycle for prevention, treatment and transmission of malaria. Re-purposing of existing FDA-approved ALDH2 inhibitors will open a rapid clinical development path for novel and effective anti-malarial drugs.

Furthermore, the use of ALDH2 inhibitors and/or aldehydic compounds as prophylactics or therapeutics is less likely to lead to drug resistance by the malaria parasite. Since the mechanism of toxicity conferred by reactive aldehydes is based on the lack of parasite ALDH function, it is unlikely that the parasite can evolve, de novo, to gain a completely new ALDH gene or a pathway for aldehyde detoxification, e.g. by gene induction or mutations in existing genes. Additionally, reactive aldehydes readily form adducts with many different macromolecules and proteins that result in cytotoxicity, a scenario in which resistance to inactivation of multiple cellular targets would be very unlikely.

### Example 2

To confirm that ALDH2*2 confers protection against malaria, the toxicity of increasing concentrations of aldehyde to malaria parasites is tested in vitro and in vivo.

Analysis of Toxicity of Aldehydes to Malaria Parasite Growth In Vitro.

A variety of in vitro cultivation methods for malaria parasites are available for screening of anti-malarial activity of chemical reagents (Trager W., et al. Human malaria parasites in continuous culture. 1976. J Parasitol. 2005; 91(3):484-6; Jambou R., et al. In vitro culture of Plasmodium berghei-ANKA maintains infectivity of mouse erythrocytes inducing cerebral malaria. Malar J. 2011; 10:346). We use well-established culture methods for parasites in RBCs (P. falciparum, P. berghei) and in hepatocytes (P. berghei) to assess the cytotoxicity of different reactive aldehydes in vitro. We focus on the potency of parasite growth inhibition for three common biogenic aldehydes: acetaldehyde (an oxidative product of ethanol metabolism), and MDA and 4HNE (two by-products of membrane lipid peroxidation). Since ALDH2 is one of the most abundant metabolic enzymes in the liver, particular attention is paid to evidence of differential susceptibility to aldehyde toxicity of parasites in hepatocytes derived from ALDH2 wild type and ALDH2*2 mice. It is expected that parasites in ALDH2*2 hepatocytes will be more susceptible to the toxic aldehydes than are parasites in ALDH2 wild type hepatocytes.

Toxicity of aldehydes in human red blood cells (RBCs). Standard procedures are used for human RBC culture with infection of late schizont/early ring stage P. falciparum for the evaluation of aldehyde toxicity. The toxicity of the above aldehydes at concentration ranges that are detectable in human blood is tested. It has been observed previously that these aldehydes do not show significant cytotoxicity to human cells at this concentration range. In brief, acetaldehyde (20 μM-1 mM), MDA (10-500 μM) and 4HNE (10-500 μM) or equivalent volumes of aldehyde diluents as controls is added to synchronized P. falciparum cultures for 48-96 hours before parasitemia is determined by flow cytometry. Sealed culture dish are used to prevent escape of volatile aldehyde vapor. Additionally, HPLC and quantitative methods are used to monitor the bioavailability of these aldehydes (Ung-Chhun N. S., et al. Estimation of blood acetaldehyde during ethanol metabolism: a sensitive HPLC/fluorescence microassay with negligible artifactual interference. Alcohol. 1987; 4(6):473-6; Budas G. R., et al. Activation of aldehyde dehydrogenase 2 (ALDH2) confers cardioprotection in protein kinase C epsilon (PKCvarepsilon) knockout mice. J Mol Cell Cardiol. 2010; 48(4):757-64), and aldehydes are provided in multiple applications as needed. Quantitative data is obtained for different doses at 48h and 96h to establish potency and IC50 of the three biogenic aldehydes. A standard hemolysis assay (Efron L., et al. Direct interaction of dermaseptin S4 aminoheptanoyl derivative with intraerythrocytic malaria parasite leading to increased specific antiparasitic activity in culture. J Biol Chem. 2002; 277(27):24067-72) is used to confirm minimal cytotoxicity of the aldehydes to human RBCs.

Toxicity of Acetaldehyde and Aldehydes to P. berghei in Established Hepatoma Cell Lines or Primary Cultures of Hepatocytes and RBCs from ALDH2 Wild Type and ALDH2*2 Mutant Mice.

Wild type or ALDH2*2 hepatoma cell lines, or primary cultures of hepatocytes are established from livers of wild type and ALDH2*2 mutant mice that we generated and infected with P. berghei sporozoites isolated from infected mosquitoes as described (Long G. W., et al. Cultivation of the exoerythrocytic stage of Plasmodium berghei in primary cultures of mouse hepatocytes and continuous mouse cell lines. In Vitro Cell Dev Biol. 1989; 25(9):857-62; Gonçalves L. A., et al. Improved isolation of murine hepatocytes for in vitro malaria liver stage studies. Malar J. 2007; 6:169; Chen C. H., et al. Cardioprotection from ischemia by a brief exposure to physiological levels of ethanol: role of epsilon protein kinase C. Proc Natl Acad Sci USA. 1999; 96(22):12784-9; Chen C., et al. Opposing effects of delta and xi PKC in ethanol-induced cardioprotection. J Mol Cell Cardiol. 2001; 33(3):581-5). Acetaldehyde, MDA and 4HNE dosing, timing, treatment and analysis of parasite development are as described above for human RBCs. Toxicity of aldehydes is tested in “prevention” mode, where aldehydes are administered to the hepatocytes prior to parasite infection, and in “treatment” mode, where aldehydes are administered to the hepatocytes after parasite infection. Evaluation of aldehyde toxicity to P. berghei is as described above for human RBCs. Alcohol as a precursor of acetaldehyde is also tested.

### Results

It is expected that no differences in aldehyde toxicity to P. berghei will be observed between cultured RBCs derived from wild type and ALDH2*2 mice, since there are no mitochondria in RBC and, hence, no ALDH2 activity in these cells (Zhang Z. W., et al. Red blood cell extrudes nucleus and mitochondria against oxidative stress. IUBMB Life. 2011; 63(7):560-5). In contrast, it is expected that parasites in the ALDH2*2 hepatocytes will exhibit reduced or no infection following aldehyde pre-treatments (prevention mode) as measured by quantitative PCR of parasite DNA. Growth-inhibition by toxic aldehydes after infection in the treatment mode is also expected. The effect should be much less pronounced in cells from wild type mice, since normal host cells can detoxify these aldehydes and thus protect the parasite. Detection of a much higher accumulation of parasite or host cell aldehydic protein adducts in the ALDH2*2.

### Example 3

To confirm that host ALDH2 enzyme deficiency confers resistance to malaria parasite infection and transmission, a murine model of malaria is used in ALDH2 deficient mice. This model is also used to detect the anti-malarial effects of pharmacological inhibitors of ALDH2 in vivo.

The rodent parasite, P. berghei, for a mosquito to mouse to mosquito infection model (Peterson T. M., et al. Nitric oxide metabolites induced in Anopheles stephensi control malaria parasite infection. Free Radic Biol Med. 2007; 42(1):132-42; Peterson T. M., et al. A mosquito 2-Cys peroxiredoxin protects against nitrosative and oxidative stresses associated with malaria parasite infection. Free Radic Biol Med. 2006; 40(6):1067-82; Cui L., et al. Molecular characterization of a prophenoloxidase cDNA from the malaria mosquito Anopheles stephensi. Insect Mol Biol. 2000; 9(2):127-37; Luckhart S., et al. The mosquito Anopheles stephensi limits malaria parasite development with inducible synthesis of nitric oxide. Proc Natl Acad Sci USA. 1998; 95(10):5700-5) is employed. In this model, infections with P. berghei ANKA and NK65 strains yield highly reproducible peripheral parasitemias and gametocytemias that are infectious to An. stephensi. The following P. berghei-infected groups are used: (1) wild type C57/BL6 mice treated with ethanol or diluent (control) at −1 day prior to infection and daily thereafter (prevention mode); (2) wild type mice treated with ethanol or diluent at 2 days post-infection and daily thereafter (treatment mode); (3) ALDH2*2 mice treated as in (1); (4) ALDH2*2 mice treated as in (2); (5) untreated, control wild type mice; and (6) untreated, control ALDH2*2 mice. The ALDH2*2 mouse is on a C57/BL6 background, a mouse strain that is the most susceptible among common mouse strains to hepatic infection with P. berghei (Scheller L. F., et al. Susceptibility of different strains of mice to hepatic infection with Plasmodium berghei. Infect Immun. 1994; 62(11):4844-7). In some instances, ALDH2 inhibitors are administered in addition to ethanol. On the day of infection, mice in each of the experimental groups are infected by bite of An. stephensi (5-10 mosquitoes/mouse). Following a 10 min feeding, salivary glands are dissected from engorged mosquitoes and scored for sporozoite density (1=1-10, 2=10-100, 3=100-1000, 4=1000+ sporozoites) so that if differences in infection patterns among mice within a treatment group are observed, the data can be stratified by relative level of parasite transmission for statistical analyses.

ALDH2 Deficient Mice are Resistant to Malaria Parasites.

During P. berghei infection, liver stages and accompanying pathology are evident by 39 hr post-infection (Scheller L. F., et al. Susceptibility of different strains of mice to hepatic infection with Plasmodium berghei. Infect Immun. 1994; 62(11):4844-7), peripheral parasitemia is evident by 4-5 days post-infection, and gametocytemia rises with peripheral parasitemia to become infectious to An. stephensi. Based on these parameters, mice that are not sacrificed within 39-48 hrs post-infection to assess liver stage parasites and pathology are examined daily for peripheral parasitemia beginning at 3 days post-infection. For ethanol treatment, both wild type and ALDH2*2 P. berghei-infected mice are challenged with 1.5 or 4.0 g/kg, or provided in drinking water (e.g. 10% ethanol) or as complete liquid diet (Lieber-DeCarli ethanol diet, Dyets, Inc. Bethlehem, Pa.)], where total liquid or calorie intake for the groups is matched (Kirpich I A, et al. The Type of Dietary Fat Modulates Intestinal Tight Junction Integrity, Gut Permeability, and Hepatic Toll-Like Receptor Expression in a Mouse Model of Alcoholic Liver Disease. Alcohol Clin Exp Res. 2012; 36(5):835-846; Wang J., et al. Ethanol induces long-term facilitation of NR2B-NMDA receptor activity in the dorsal striatum: implications for alcohol drinking behavior. J Neurosci. 2007; 27(13):3593-602). Elevated blood acetaldehyde levels are confirmed by HPLC from blood samples collected under anesthesia by retro-orbital puncture from a parallel set of matched treatment and control mice. Replicated sets of mice from these treatments and controls are examined for exoerythrocytic parasite development (39-48 hours post-infection; scored as parasites per liver) (Scheller L. F., et al. Susceptibility of different strains of mice to hepatic infection with Plasmodium berghei. Infect Immun. 1994; 62(11):4844-7) and liver pathology (39-48 hours post-infection; scored as inflammatory infiltrates, microabscesses, granulomas) (Scheller L. F., et al. Susceptibility of different strains of mice to hepatic infection with Plasmodium berghei. Infect Immun. 1994; 62(11):4844-7; Roux C. M., et al. Both hemolytic anemia and malaria parasite-specific factors increase susceptibility to Nontyphoidal Salmonella enterica serovar typhimurium infection in mice. Infect Immun. 2010; 78(4):1520-7). Erythrocytic parasite development is determined based on daily peripheral parasitemia and gametocytemia as well as infectivity to An. stephensi. For the latter, infected mice will be anesthetized and exposed to 20-30 mosquitoes for 10-15 min. After feeding, mice are euthanized under anesthesia and necropsied for tissue samples (blood, liver, spleen, brain) for histopathology and assessment of infection. Blood fed mosquitoes are maintained for quantification of P. berghei oocyst development (7 days post-feeding) and sporozoite infection (12 days post-feeding) according to standard protocols in the Luckhart lab. Mouse blood and tissue samples are also analyzed for the presence or elevated levels of aldehyde-protein adduct formation by immunoblot.

Significant differences among treatment and control mice are analyzed by ANOVA followed by Student-Neuman-Keuls for means separation or by Kruskal-Wallis followed by Dunn's post-test for means separation for non-normally distributed data. Data is analyzed as mosquito intensity of infection (mean oocysts or sporozoite scores) and infection prevalence (the occurrence of mosquitoes with at least one oocyst or detectable sporozoites) across treatment groups and controls. Effects of mouse and treatment group is determined by two-way ANOVA or Friedman's test followed by appropriate analyses.

The Anti-Malarial Effects of Pharmacological Inhibitors of ALDH2 In Vivo.

Disulfiram (Antabuse®) and diadzin are administered to wild type C57/BL6 mice using subcutaneously implanted osmotic pumps (Alzet, Cupertino, Calif.) as described in [45, 46]. The dose of these inhibitors is calibrated based on published pharmacokinetics data. The efficacy of these inhibitors is confirmed by measuring ALDH enzyme activity in liver tissues from treated animals. In this study. control mice and mice treated with disulfiram or diadzin are treated in a prevention or treatment mode as described above. P. berghei infection, ethanol dosing, monitoring of blood acetaldehyde concentration, parasitemia counts, histopathology and mosquito transmission studies are monitored as described above.

### Results

Based on preliminary results, it is expected that 1.5 to 4.0 g/kg of acute ethanol administration will produce at least 5 fold difference in blood acetaldehyde level between the wild type and ALDH2*2 mutant animals. This difference is sustained for at least 4-6 hours after single dose of ethanol. Among wild type mice, no significant differences in parasite infection, histopathology, or transmission due to ethanol treatment is expected, since ethanol-derived acetaldehyde at this dose is quickly metabolized. In contrast, a significant resistance to parasite infection, development and/or transmission is expected in the ALDH2*2 mutants, which is enhanced in mutant mice receiving ethanol relative to the respective controls. In addition, a dose effect of ethanol treatment on the inhibition of parasite infection and transmission is expected.

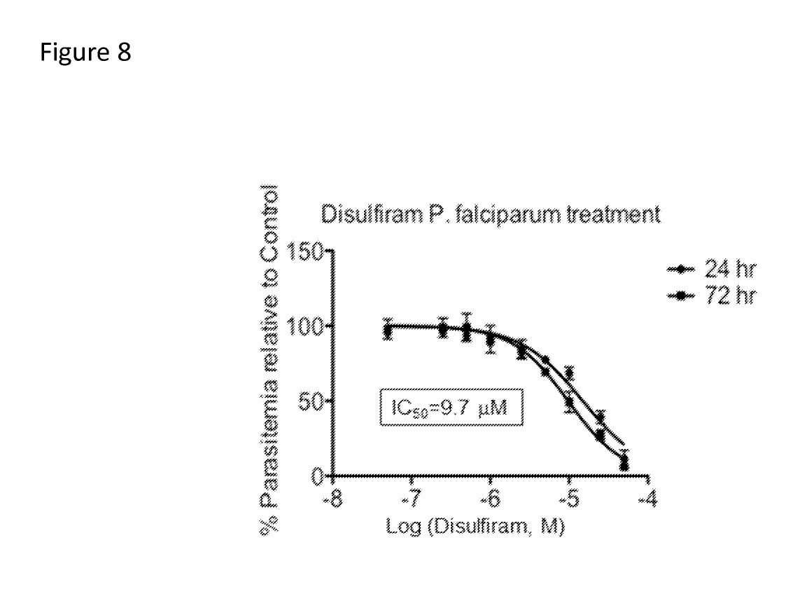

Disulfirm and diadzin are used as deterrents for treatment of alcoholism, and as such, are both safe and effective ALDH2 inhibitors. It is expected that the suppression of ALDH2 during ethanol treatment will increase acetaldehyde levels, thus reducing parasite infection and development that is similar to that expected in the ALDH2*2 mice. These data will confirm that ALDH2 is a valid target for anti-malarial drug development. Our results indicated that ALDH inhibitor, disulfiram, significantly inhibited parasite growth (IC₅₀=9.7 μM) in human RBC culture in the absence of alcohol, by inhibiting the cytosolic ALDH activity in RBC (FIG. 8). As a negative control, pyrazole, an alcohol dehydrogenase inhibitor, at a concentration as high as 500 μM has no effect on the growth of parasites.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for treating a parasitic disease in a subject having a parasitic disease, comprising:
administering to the subject an effective amount of an Aldehyde Dehydrogenase (ALDH) antagonist,
wherein the parasitic disease is treated.

2. The method according to claim 1, wherein the ALDH antagonist is a small molecule.

3. The method according to claim 1, wherein the ALDH antagonist is a nucleic acid.

4. The method according to claim 1, wherein the ALDH antagonist is a protein.

5. The method according to claim 1, wherein the ALDH antagonist inhibits ALDH2 activity.

6. The method according to claim 1, further comprising administering an effective amount one or more additional anti-parasitic agents.

7. The method according to claim 6, wherein the one or more additional anti-parasitic agents is an aldehyde.

8. The method according to claim 7, wherein the aldehyde is selected from the group consisting of acetaldehyde and 4HNE.

9. The method according to claim 1, wherein the parasitic disease is associated with infection by a protozoan.

10. The method according to claim 9, wherein the protozoan is a hematozoan.

11. The method according to claim 10, wherein the hematozoan is a Plasmodium.

12. The method according to claim 10, wherein hematozoan is a Babesia.

13. The method according to claim 10, wherein the hematozoan is a Theileria.

14. The method according to claim 1, wherein the subject is a human.

15. A method for preventing a parasitic disease in a subject at risk for developing a parasitic disease, comprising:
administering to the subject an effective amount of ALDH antagonist,
wherein the parasitic disease is prevented.

16. The method according to claim 15, wherein the method further comprises administering an aldehyde.

17. The method according to claim 15, wherein the subject is infected by or at risk for becoming infected by a parasite that causes the parasitic disease.

18. The method according to claim 17, wherein the parasite is a protozoan.

19. The method according to claim 18, wherein the protozoan is a hematozoan.

20. The method according to claim 19, wherein the hematozoan is a Plasmodium.

21. The method according to claim 19, wherein hematozoan is a Babesia.

22. The method according to claim 19, wherein the hematozoan is a Theileria.

23. The method according to claim 15, wherein the subject is a human.

24. A kit for the treatment or prevention of a parasitic disease, comprising an ALDH antagonist.

25. The kit according to claim 24, further comprising an aldehyde.

26. The kit according to claim 25, wherein the aldehyde is selected from the group consisting of acetaldehyde and 4HNE.

27. The kit according to claim 24, further comprising a reagent or device for detecting a parasite associated with the parasitic disease.

28. The kit according to claim 24, wherein the parasite is from the genus Plasmodium, Babesia, or Theileria.
